SEQUENCE LISTING (9 sequences)

SEQ ID NO: 1 - DNA, 21 bp, Synthetic construct
  source 1..21: mol_type = other DNA
ggtatgacccgaggtgtaatg

SEQ ID NO: 2 - DNA, 22 bp, synthetic construct
  source 1..22: mol_type = other DNA
ctaccagctcacacactgcatc

SEQ ID NO: 3 - DNA, 20 bp, synthetic construct
  source 1..20: mol_type = other DNA
catggcaatttagccgattg

SEQ ID NO: 4 - DNA, 20 bp, synthetic construct
  source 1..20: mol_type = other DNA
gaaacaaactgacctcgagc

SEQ ID NO: 5 - DNA, 20 bp, synthetic construct
  source 1..20: mol_type = other DNA
catgatggtctttatggtgc

SEQ ID NO: 6 - DNA, 19 bp, synthetic construct
  source 1..19: mol_type = other DNA
cagtttcaccacctgctgc

SEQ ID NO: 7 - DNA, 21 bp, synthetic construct
  source 1..21: mol_type = other DNA
ggtattgatcgtcggcttaag

SEQ ID NO: 8 - DNA, 20 bp, synthetic construct
  source 1..20: mol_type = other DNA
caatatcttgatatgggttc

SEQ ID NO: 9 - DNA, 18 bp, synthetic construct
  source 1..18: mol_type = other DNA
caactgccgaccgctatc

(19) United States
(12) Patent Application Publication
(10) Pub. No.: US 2025/0051300 A1
(43) Pub. Date: Feb. 13, 2025

(54) GRAM-NEGATIVE SPECIFIC ANTIBIOTICS SPARING EFFECT ON GUT MICROBIOME

(71) Applicants: Paul J. HERGENROTHER, Champaign, IL (US); Kristen A. MUÑOZ, Urbana, IL (US); THE BOARD OF TRUSTEES OF THE UNIVERSITY OF ILLINOIS, Urbana, IL (US)

(72) Inventors: Paul J. HERGENROTHER, Champaign, IL (US); Kristen A. MUÑOZ, Urbana, IL (US)

(73) Assignee: THE BOARD OF TRUSTEES OF THE UNIVERSITY OF ILLINOIS, Urbana, IL (US)

(21) Appl. No.: 18/710,813

(22) PCT Filed: Nov. 10, 2022
(86) PCT No.: PCT/US2022/079610

§ 371 Date: May 16, 2024

Related U.S. Application Data
(60) Provisional application No. 63/281,475, filed on Nov. 19, 2021.

(51) Int. Cl. C07D 401/04; A61K 31/4439; A61P 31/04

(52) U.S. Cl. CPC C07D 401/04; A61K 31/4439; A61P 31/04

(57) ABSTRACT

Small molecule inhibitors of the localization of lipoprotein CDE (LolCDE) complex that is found in the membrane of bacteria. Searches for suitable candidates for the LolCDE complex led to the discovery of an inhibitor named lolamycin. Lolamycin specifically targets Gram-negative bacteria such as Escherichia coli, Klebsiella pneumoniae, Enterobacter cloacae, and Salmonella typhimurium, and is selective over Gram-negative and Gram-positive commensal bacteria, thereby avoiding perturbation of the gut microbiome.

### RELATED APPLICATIONS

This application claims priority under 35 U.S.C. § 119(e) to U.S. Provisional Patent Application No. 63/281,475, filed Nov. 19, 2021, which is incorporated herein by reference.

### GOVERNMENT SUPPORT

This invention was made with government support under Grant No. 5R01AI136773 awarded by the National Institutes of Health. The government has certain rights in the invention.

### BACKGROUND OF THE INVENTION

The gut microbiome plays a significant role in host health, affecting nutrition, digestion, metabolism, and immune response. Perturbation of the microbiome resulting from antibiotic treatment can lead to detrimental effects including increased vulnerability to colonization of opportunistic pathogens, like Clostridioides difficile, as well as increased risk for gastrointestinal, renal, and hematological abnormalities. The vast majority of clinically approved antibiotics only kill Gram-positive bacteria (Gram-positive-only antibiotics) or kill both Gram-positive and Gram-negative bacteria (broad-spectrum antibiotics). Strikingly, surveys assessing large numbers of different antibiotics against a variety of commensal bacteria have shown that essentially all these drugs have a deleterious effect on gut bacteria. Gut dysbiosis has been linked to both Gram-positive-only and broad-spectrum antibiotics. Extensive studies reveal that fluoroquinolone treatment results in rapid and significant decreases in taxonomic richness and diversity, while other broad-spectrum classes (cephalosporins and ß-lactams) are associated with profound losses of intestinal symbionts. Even short exposure to Gram-positive-only clindamycin results in long-lasting changes, affecting taxonomic composition and selection for resistance genes, detectable up to two years after dosing. Currently, the effect of Gram-negative-only antibiotics on microbiome perturbation remains unclear as there are vanishingly few of these compounds. And, they are challenging to discover as the majority of potential antibiotic targets are shared between Gram-positive and Gram-negative bacteria. A rare Gram-negative-only antibiotic that is clinically approved, colistin, has antibacterial activity against some human gut commensals in addition to being nephrotoxic. Thus, the ability to assess the hypothesis that Gram-negative-only antibiotics can be gut microbiome sparing is limited by the current antibiotic arsenal.

With a rise in difficult-to-treat Gram-negative infections and the increased global emergence of colistin-resistant genes in Enterobacterales species, the need for novel antibacterials (especially for Gram-negative ESKAPE pathogens) is critical. However, due to the intricate cellular membrane structure and embedded efflux pumps within Gram-negative bacteria, the discovery of antibiotics active against these pathogens is difficult, and as a result no new class of antibiotics active against Gram-negative bacteria have been approved by the Food and Drug Administration (FDA) in over 50 years. The discovery of antibiotics selective for Gram-negative bacteria is even more arduous; targets in the outer membrane could be exploitable, but most such compounds identified only have activity against Gram-negative strains whose membrane has been compromised. As such, activity versus wild-type Gram-negatives and clinical isolates remains elusive, with the recent discovery of BamA-targeting natural products a notable exception (Nature 576, 459-464 (2019)).

The gut microbiome consists of both Gram-positive and Gram-negative bacteria, so even an antibiotic specific for Gram-negative bacteria would be expected to induce gut dysbiosis. Key to developing a Gram-negative-only antibiotic with minimal disturbance to the microbiome is to target a protein or complex exclusively present in Gram-negatives and specific for pathogens over commensals. An underexplored cellular target for development of such narrow-spectrum drugs is the Lol system, a five-part protein system located in the periplasm that is responsible for lipoprotein transport across the inner and outer membranes in Gram-negative bacteria (Biochim. Biophys. Acta 1693, 5-13 (2004)). Since lipoproteins of Gram-positive bacteria are entirely retained and anchored to the cytoplasmic membrane, lipoprotein transport is not required, making the Lol system exclusive to Gram-negatives. This lipoprotein transfer system is shown to be crucial for the growth of Escherichia co/i and is conserved across high-priority Gram-negative ESKAPE pathogens. Notably, as detailed later, the Lol system of E. coli shares low sequence homology with human commensal Gram-negatives, suggesting the potential for exploiting this protein complex as a means to develop doubly-selective antibiotics: a Gram-negative-only target with further specificity for Gram-negative pathogens.

The problem is the lack of alternative treatments for Gram-negative infections and the gut dysbiosis that occurs with antibacterial treatments. Accordingly, there is a need for antibiotics that target bacterial infections through different mechanisms of action.

### SUMMARY

This disclosure provides a an LolCDE inhibitor with antibiotic activity against wild-type Gram-negative pathogens. The compounds are Gram-negative-only antibiotics that also act as microbiome-sparing drugs. Herein is reported the compound lolamycin, an antibiotic acting through disruption of the Lol system. Lolamycin has tremendous specificity for certain Gram-negative ESKAPE pathogens over commensal bacteria and is active in mouse infection models while sparing the gut microbiome. The features of lolamycin can avoid the onset of a C. difficile infection from treatment.

Accordingly, this disclosure provides a compound of Formula I:

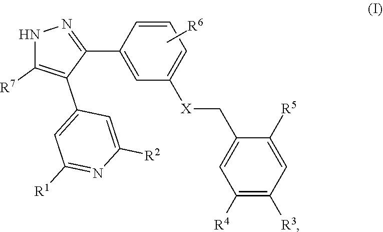

or a salt thereof,
wherein
R¹ and R² are each independently —CH₃, —(C₂-C₆)alkyl or —(C₃-C₆)cycloalkyl, or H;
R³ is H, halo, cyano, —(C₁-C₆)alkyl, or —(C₃-C₆)cycloalkyl;
R⁴ is cyano, H, halo, —(C₁-C₆)alkyl, or —(C₃-C₆)cycloalkyl;
R⁵ and R⁶ are each independently H, halo, cyano, —(C₁-C₆)alkyl, or —(C₃-C₆)cycloalkyl; and
R⁷ is H, —(C₁-C₆)alkyl, or —(C₃-C₆)cycloalkyl; and
X is O, S, NH, or NCH₃;
   wherein any methyl, alkyl, or cycloalkyl moiety is optionally substituted; provided that when R¹ and R² are both H, R³ is not F and R³, R⁴, and R⁵ are not all H.

This disclosure also provides a method for treating a patient infected with Gram-negative bacteria comprising administering to the patient a therapeutically effective amount of a compound described herein or a pharmaceutical composition thereof, wherein the infected patient is thereby treated.

The invention provides novel compounds of Formula I and Formula II, intermediates for the synthesis of compounds of Formula I and Formula II, as well as methods of preparing compounds of Formula I and II. The invention also provides compounds of Formula I and II that are useful as intermediates for the synthesis of other useful compounds. The invention provides for the use of compounds of Formula I and Formula II for the manufacture of medicaments useful for the treatment of bacterial infections in a mammal, such as a human.

The invention provides for the use of the compositions described herein for use in medical therapy. The medical therapy can be treating a bacterial infection, for example, an infection with Gram-negative bacteria. The invention also provides for the use of a composition as described herein for the manufacture of a medicament to treat a bacterial infection in a mammal, for example, an infection from Escherichia coli, Klebsiella pneumoniae, Enterobacter cloacae, or Salmonella typhimurium in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Diagram for targeting the lipoprotein transport pathway as a means of developing narrow-spectrum agents.

FIG. 2. (A) eNTRy rule parameters (Rotatable bonds, Globularity, Functional Group) of compounds 1 and 2 calculated using eNTRyway. Accumulation determined via an accumulation assay. Data shown represents the average of three independent experiments with standard deviation of the mean. (B) Accumulation of amine-containing derivatives (compounds 3-8) determined via an accumulation assay. Data shown represents the average of three independent experiments with standard deviation of the mean. MIC values were determined using the micro-dilution broth method as outlined by the Clinical and Laboratory Standards Institute (CLSI. Methods for Dilution Antimicrobial Susceptibility Tests for Bacteria that Grow Aerobically. 11^{th} ed. CLSI standard M07. Wayne, PA: Clinical and Laboratory Standards Institute; 2018) and are listed in μg/mL. All experiments were performed in biological triplicate.

FIG. 3. Assessment of compound 2 and lolamycin against MDR clinical isolates of (A) E. coli (n=46), (B) K. pneumoniae (n=61), and (C) E. cloacae (n=18). MICs were performed in MH Broth per CLSI guidelines. See Table 8 for a list of resistance genes in clinical isolate panels. A full listing of this MIC data is in Table 9-11.

FIG. 4. (A) Frequency of E. coli JW5503 ΔtolC resistance to compound 2 and lolamycin as a function of concentration. (B) Frequency of E. coli BW25113, K. pneumoniae ATCC 27736, and E. cloacae ATCC 29893 resistance to lolamycin as a function of concentration.

FIG. 5. Growth rates of Lolamycin resistant mutants generated in E. coli BW25113 vs wild-type (WT) E. coli BW25113. Fitness of E. coli isolates that harbor the mutations in LolC (A) or LolE (B) conferring resistance to Lolamycin at 32-fold above the MIC (64 μg/mL) were evaluated for growth in culture relative to the parental strain. Bacterial growth was performed in cation-adjusted MH broth. Each mutant and the parental strain were assessed in biological triplicate. Measurements were compared by repeated-measures two-way analysis of variance (ANOVA). Tukey's multiple comparisons test was used to compare strains at each time point. After measuring growth rates and the final OD at stationary phase of wild-type and lolamycin resistant mutants, no statistically significant difference was observed. NS, not significant.

FIG. 6. (A) Lolamycin binding sites overlaid with modeled lipoprotein. Modeled lipoprotein shown in licorice representation while lolamycin in BS1, BS2, TS1, and TS2 are shown as space filling structures right, left, middle, and top respectively. (B) High probability residue contacts (>0.2 probability) for each binding/transient site. Cutoff used for calculation was 3.5 Å.

FIG. 7. (A) Bacterial burden model of acute pneumonia in E. coli. Acute pneumonia infections initiated in 7-week-old CD-1 mice with E. coli AR0349 (2.7×10⁸ CFUs/mouse intranasally). Mice were treated with vehicle (n=8) or compound (2 or lolamycin, 100 mg/kg IP, n=8 for each group) 4, 8, 24, 32, 48, and 56 h post-infection, and the bacterial burden was evaluated 72 h post-infection. MIC=1 μg/mL for Lolamycin. (B) Kaplan-Meier survival curve of the mouse efficacy model of E. coli sepsis. Seven-week-old CD-1 mice were infected with E. coli AR0349 (4.2×10⁸ CFUs/mouse; n=15 per group) via retro-orbital injection. Mice were treated with compound (2 or lolamycin, 100 mg/kg IP, n=15 for each group) twice daily post-infection for 3 days. (C) Bacterial burden model of acute pneumonia in K. pneumoniae. Acute pneumonia infections initiated in 7-week-old CD-1 mice with K. pneumoniae AR0040 (8×10⁷ CFUs/mouse intranasally). Mice were treated with vehicle (n=8) or compound (2 or lolamycin, 100 mg/kg IP, n=8 for each group) 4, 10, 21, 29, 43, and 51 h post-infection, and the bacterial burden was evaluated 72 h post-infection. MIC=1 μg/mL for lolamycin. (D) Kaplan-Meier survival curve of the mouse efficacy model of K. pneumoniae sepsis. Seven-week-old CD-1 mice were infected with K. pneumoniae BAA-1705 (5.8×10⁷ CFUs/mouse; 15 mice per group) via retro-orbital injection. Mice were treated with the compound (2 or lolamycin, 200 mg/kg IP, n=15 for each group) once daily post-infection for 3 days. (E) Bacterial burden model of acute pneumonia in E. cloacae. Acute pneumonia infections initiated in 7-week-old CD-1 mice with E. cloacae AR0163 (7.2×10⁸ CFUs/mouse intranasally). Mice were treated with vehicle (n=8) or compound (2 or lolamycin, 100 mg/kg IP, n=8 for each group) 4, 11, 24, 32, 48, and 56 h post-infection, and the bacterial burden was evaluated 72 h post-infection. MIC=8 μg/mL for lolamycin. (F) Kaplan-Meier survival curve of the mouse efficacy model of E. cloacae sepsis. Seven-week-old CD-1 mice were infected with E. cloacae AR0163 (8.95×10⁸ CFUs/mouse; 15 mice per group) via retro-orbital injection. Mice were treated with compound (2 or lolamycin, 100 mg/kg IP, n=15 for each group) twice daily post-infection for 3 days. In A, C, and E, data are shown as means±s.d. and statistical significance was determined by one-way ANOVA with Tukey's multiple comparisons. In B, D, and F, statistical significance was determined by two-tailed log-rank (Mantel-Cox) test. NS=not significant (P>0.05), ***P<0.001, ****P<0.0001.

FIG. 8. (A) Bacterial burden model of acute pneumonia in E. coli. Acute pneumonia infections initiated in 7-week-old CD-1 mice with E. coli AR0349 (2.9×10⁸ CFUs/mouse intranasally). Mice were treated with vehicle (n=8) or compound (lolamycin, 200 mg/kg PO, n=8) and the bacterial burden was evaluated 72 h post-infection. MIC=1 μg/mL for Lolamycin. (B) Kaplan-Meier survival curve of the mouse efficacy model of E. coli sepsis. Seven-week-old CD-1 mice were infected with E. coli AR0349 (6.0×10⁸ CFUs/mouse) via retro-orbital injection. Mice were treated with vehicle (n=15) or compound (lolamycin, 200 mg/kg PO, n=15) twice daily post-infection for 3 days. In A, data are shown as means±s.d. and statistical significance was determined by one-way ANOVA with Tukey's multiple comparisons. In B, statistical significance was determined by two-tailed log-rank (Mantel-Cox) test. ****P<0.0001.

FIG. 9. Alpha diversity analysis before (Day 0) and four days after (Day 7) antibiotic administration measured by Shannon Index. **=p<0.01.

FIG. 10. Rarefaction analysis showing the species richness as sequencing progressed throughout antibiotic treatment and recovery period (Day 0, Day 7, Day 10, Day 31).

### DETAILED DESCRIPTION

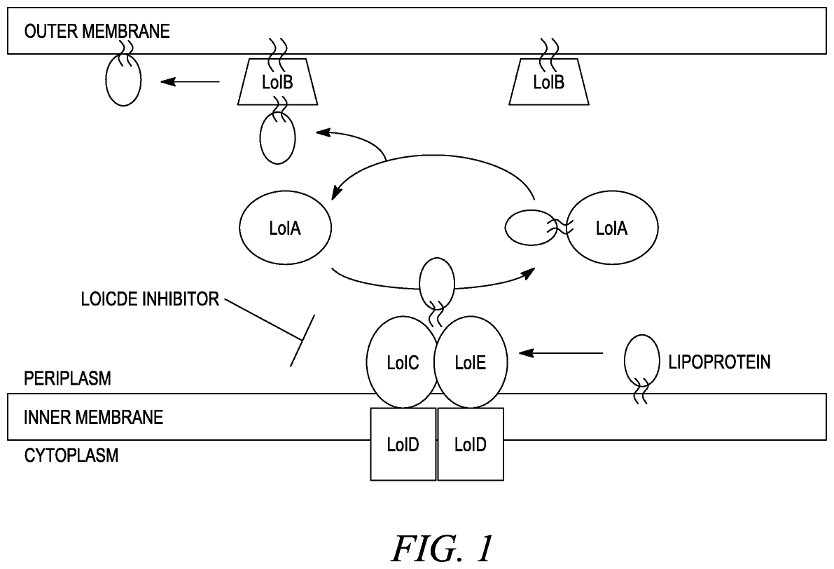

Minimal perturbation of the gut microbiome resulting from treatment with lolamycin highlights an improved strategy for developing Gram-negative-only antibiotics. Targeting the Lol system (FIG. 1) proves advantageous as this complex is highly conserved across pathogens but shows low sequence homology amongst gut symbionts and as such, discovery of Lol inhibitors has garnered increasing attention over the years (Antimicrob. Agents Chemother. 62, (2018)). As microbiome-sparing potential will likely become a desirable trait in antibiotic development, leveraging this lipoprotein transport system for designing selective inhibitors is desirable.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood by an ordinary person skilled in the art that a range, such as “number1” to “number2”, implies a continuous range of numbers that includes the whole numbers and fractional numbers. For example, 1 to 10 means 1, 2, 3, 4, 5, . . . 9, 10. It also means 1.0, 1.1, 1.2, 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

The recitation of a), b), c), . . . or i), ii), iii), or the like in a list of components or steps do not confer any particular order unless explicitly stated.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, a patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The compound and compositions described herein may be administered with additional compositions to prolong stability and activity of the compositions, or in combination with other therapeutic drugs.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

This disclosure provides methods of making the compounds and compositions of the invention. The compounds and compositions can be prepared by any of the applicable techniques described herein, optionally in combination with standard techniques of organic synthesis. Many techniques such as etherification and esterification are well known in the art. However, many of these techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6; as well as standard organic reference texts such as March's Advanced Organic Chemistry: Reactions, Mechanisms, and Structure, 5th Ed., by M. B. Smith and J. March (John Wiley & Sons, New York, 2001); Comprehensive Organic Synthesis. Selectivity, Strategy & Efficiency in Modem Organic Chemistry. In 9 Volumes, Barry M. Trost, Editor-in-Chief (Pergamon Press, New York, 1993 printing); Advanced Organic Chemistry, Part B: Reactions and Synthesis, Second Edition, Cary and Sundberg (1983); for heterocyclic synthesis see Hermanson, Greg T., Bioconjugate Techniques, Third Edition, Academic Press, 2013.

The formulas and compounds described herein can be modified using protecting groups. Suitable amino and carboxy protecting groups are known to those skilled in the art (see for example, Protecting Groups in Organic Synthesis, Second Edition, Greene, T. W., and Wutz, P. G. M., John Wiley & Sons, New York, and references cited therein; Philip J. Kocienski; Protecting Groups (Georg Thieme Verlag Stuttgart, New York, 1994), and references cited therein); and Comprehensive Organic Transformations, Larock, R. C., Second Edition, John Wiley & Sons, New York (1999), and referenced cited therein.

The term “halo” or “halide” refers to fluoro, chloro, bromo, or iodo. Similarly, the term “halogen” refers to fluorine, chlorine, bromine, and iodine.

The term “alkyl” refers to a branched or unbranched hydrocarbon having, for example, from 1-20 carbon atoms, and often 1-12, 1-10, 1-8, 1-6, or 1-4 carbon atoms; or for example, a range between 1-20 carbon atoms, such as 2-6, 3-6, 2-8, or 3-8 carbon atoms. As used herein, the term “alkyl” also encompasses a “cycloalkyl”, defined below. Examples include, but are not limited to, methyl, ethyl, 1-propyl, 2-propyl (iso-propyl), 1-butyl, 2-methyl-1-propyl (isobutyl), 2-butyl (sec-butyl), 2-methyl-2-propyl (t-butyl), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, hexyl, octyl, decyl, dodecyl, and the like. The alkyl can be unsubstituted or substituted, for example, with a substituent described below or otherwise described herein. The alkyl can also be optionally partially or fully unsaturated. As such, the recitation of an alkyl group can include an alkenyl group or an alkynyl group. The alkyl can be a monovalent hydrocarbon radical, as described and exemplified above, or it can be a divalent hydrocarbon radical (i.e., an alkylene).

An alkylene is an alkyl group having two free valences at a carbon atom, or two different carbon atoms, of a carbon chain. Similarly, alkenylene and alkynylene are respectively an alkene and an alkyne having two free valences at a carbon atom or two different carbon atoms.

The term “cycloalkyl” refers to cyclic alkyl groups of, for example, from 3 to 10 carbon atoms having a single cyclic ring or multiple condensed rings. Cycloalkyl groups include, by way of example, single ring structures such as cyclopropyl, cyclobutyl, cyclopentyl, cyclooctyl, and the like, or multiple ring structures such as adamantyl, and the like. The cycloalkyl can be unsubstituted or substituted. The cycloalkyl group can be monovalent or divalent, and can be optionally substituted as described for alkyl groups. The cycloalkyl group can optionally include one or more cites of unsaturation, for example, the cycloalkyl group can include one or more carbon-carbon double bonds, such as, for example, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

The term “heteroatom” refers to any atom in the periodic table that is not carbon or hydrogen. Typically, a heteroatom is O, S, N, P. The heteroatom may also be a halogen, metal or metalloid.

The term “heterocycloalkyl” or “heterocyclyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morpholino, 1,3-diazapane, 1,4-diazapane, 1,4-oxazepane, and 1,4-oxathiapane. The group may be a terminal group or a bridging group.

The term “aromatic” refers to either an aryl or heteroaryl group or substituent described herein. Additionally, an aromatic moiety may be a bisaromatic moiety, a trisaromatic moiety, and so on. A bisaromatic moiety has a single bond between two aromatic moieties such as, but not limited to, biphenyl, or bipyridine. Similarly, a trisaromatic moiety has a single bond between each aromatic moiety.

The term “aryl” refers to an aromatic hydrocarbon group derived from the removal of at least one hydrogen atom from a single carbon atom of a parent aromatic ring system. The radical attachment site can be at a saturated or unsaturated carbon atom of the parent ring system. The aryl group can have from 6 to 30 carbon atoms, for example, about 6-10 carbon atoms. The aryl group can have a single ring (e.g., phenyl) or multiple condensed (fused) rings, wherein at least one ring is aromatic (e.g., naphthyl, dihydrophenanthrenyl, fluorenyl, or anthryl). Typical aryl groups include, but are not limited to, radicals derived from benzene, naphthalene, anthracene, biphenyl, and the like. The aryl can be unsubstituted or optionally substituted with a substituent described below. For example, a phenyl moiety or group may be substituted with one or more substituents Rx where Rx is at the ortho-, meta-, orpara-position, and X is an integer variable of 1 to 5.

The term “heteroaryl” refers to a monocyclic, bicyclic, or tricyclic ring system containing one, two, or three aromatic rings and containing at least one nitrogen, oxygen, or sulfur atom in an aromatic ring. The heteroaryl can be unsubstituted or substituted, for example, with one or more, and in particular one to three, substituents, as described in the definition of “substituted”. Typical heteroaryl groups contain 2-20 carbon atoms in the ring skeleton in addition to the one or more heteroatoms, wherein the ring skeleton comprises a 5-membered ring, a 6-membered ring, two 5-membered rings, two 6-membered rings, or a 5-membered ring fused to a 6-membered ring. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, chromenyl, cinnolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, tetrazolyl, and xanthenyl. In one embodiment the term “heteroaryl” denotes a monocyclic aromatic ring containing five or six ring atoms containing carbon and 1, 2, 3, or 4 heteroatoms independently selected from non-peroxide oxygen, sulfur, and N(Z) wherein Z is absent or is H, O, alkyl, aryl, or (C₁-C₆)alkylaryl. In some embodiments, heteroaryl denotes an ortho-fused bicyclic heterocycle of about eight to ten ring atoms derived therefrom, particularly a benz-derivative or one derived by fusing a propylene, trimethylene, or tetramethylene diradical thereto.

As used herein, the term “substituted” or “substituent” is intended to indicate that one or more (for example, in various embodiments, 1-10; in other embodiments, 1-6; in some embodiments 1, 2, 3, 4, or 5; in certain embodiments, 1, 2, or 3; and in other embodiments, 1 or 2) hydrogens on the group indicated in the expression using “substituted” (or “substituent”) is replaced with a selection from the indicated group(s), or with a suitable group known to those of skill in the art, provided that the indicated atom's normal valency is not exceeded, and that the substitution results in a stable compound. Suitable indicated groups include, e.g., alkyl, alkenyl, alkynyl, alkoxy, haloalkyl, hydroxyalkyl, aryl, heteroaryl, heterocyclyl, cycloalkyl, alkanoyl, alkoxycarbonyl, amino, alkylamino, dialkylamino, carboxyalkyl, alkylthio, alkylsulfinyl, and alkylsulfonyl. Substituents can be the indicated groups, one of a specific list of substituents described herein, or as one of skill in the art would recognize, they can be one or more substituents selected from alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, and cyano. Suitable substituents that can be bonded to a substituted carbon atom also include F, Cl, Br, I, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety (e.g., (C₁-C₆)alkyl), and wherein the carbon-based moiety can itself be further substituted. When a substituent is monovalent, such as, for example, F or Cl, it is bonded to the atom it is substituting by a single bond. When a substituent is divalent, such as 0, it is bonded to the atom it is substituting by a double bond; for example, a carbon atom substituted with 0 forms a carbonyl group, C═O.

The term “IC₅₀” is generally defined as the concentration required to inhibit a specific biological or biochemical function by half, or to kill 50% of the cells in a designated time period, typically 24 hours.

### STATEMENTS OF THE INVENTION

1. A compound of Formula I:

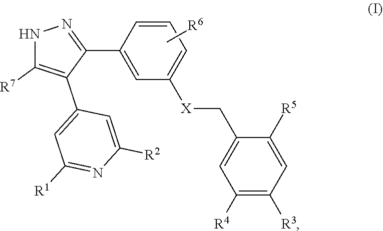

or a salt thereof;
wherein
R¹ and R² are each independently —CH₃, —(C₂-C₆)alkyl or —(C₃-C₆)cycloalkyl, or H;
R³ is H, halo, cyano, —(C₁-C₆)alkyl, or —(C₃-C₆)cycloalkyl;
R⁴ is cyano, H, halo, —(C₁-C₆)alkyl, or —(C₃-C₆)cycloalkyl;
R⁵ and R⁶ are each independently H, halo, cyano, —(C₁-C₆)alkyl, or —(C₃-C₆)cycloalkyl; and
R⁷ is H, —(C₁-C₆)alkyl, or —(C₃-C₆)cycloalkyl; and
X is O, S, NH, or NCH₃;
   wherein any methyl (—CH₃), alkyl, or cycloalkyl moiety is optionally substituted; provided that when R¹ and R² are both H, R³ is not F and R³, R⁴, and R⁵ are not all H.

2. The compound of statement 1 wherein X is O.

3. The compound of statement 1 or 2 wherein R¹ and R² are CH₃.

4. The compound of any one of statements 1-3 wherein R³ is H, F, CN, or CH₂NH₂ (an example of C₁ alkyl substituted by an amino (NH₂) substituent).

5. The compound of any one of statements 1-4 wherein R⁴ is H, CN, or CH₂NH₂.

6. The compound of any one of statements 1-5 wherein R⁵ is H, CN, or CH₂NH₂.

7. The compound of any one of statements 1-6 wherein R⁶ is H, CN, or CH₂NH₂.

8. The compound of any one of statements 1-7 wherein R⁶ is at the position para to the pyrazole of Formula I.

9. The compound of any one of statements 1-8 wherein R⁷ is H.

10. The compound of any one of statements 1-9 wherein one or two of R³, R⁴ and R⁵ are H.

11. The compound of any one of statements 1-10 wherein one of the alkyl or cycloalkyl moieties have an amine substituent.

12. The compound of statement 1 represented by Formula II:

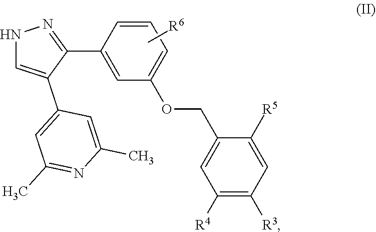

or a salt thereof.

13. The compound of statement 1 wherein the compound is lolamycin:

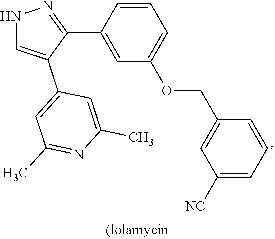 [SMILES: Cc1cc(-c2c[nH]nc2-c2cccc(OCc3cccc(C#N)c3)c2)cc(C)n1]

or a salt thereof.

14. The compound of statement 1 wherein the compound is:
(4-((3-(4-(pyridin-4-yl)-1H-pyrazol-3-yl)phenoxy)methyl)phenyl)methanamine (3);
(4-((3-(4-(2,6-dimethylpyridin-4-yl)-1H-pyrazol-3-yl)phenoxy)methyl)phenyl)methanamine (4);
(3-((3-(4-(2,6-dimethylpyridin-4-yl)-1H-pyrazol-3-yl)phenoxy)methyl)phenyl)methanamine (5);
(2-((3-(4-(pyridin-4-yl)-1H-pyrazol-3-yl)phenoxy)methyl)phenyl)methanamine (6);
(2-((3-(4-(2,6-dimethylpyridin-4-yl)-1H-pyrazol-3-yl)phenoxy)methyl)phenyl)methanamine (7);
(4-(4-(2,6-dimethylpyridin-4-yl)-1H-pyrazol-3-yl)-2-((4-fluorobenzyl)oxy)phenyl)methanamine (8);
4-(3-(3-((4-fluorobenzyl)oxy)phenyl)-1H-pyrazol-4-yl)-2,6-dimethylpyridine (9);
4-((3-(4-(pyridin-4-yl)-1H-pyrazol-3-yl)phenoxy)methyl)benzonitrile (10);
4-((3-(4-(2,6-dimethylpyridin-4-yl)-1H-pyrazol-3-yl)phenoxy)methyl)benzonitrile (11);
3-((3-(4-(2,6-dimethylpyridin-4-yl)-1H-pyrazol-3-yl)phenoxy)methyl)benzonitrile (12, lolamycin);
2-((3-(4-(pyridin-4-yl)-1H-pyrazol-3-yl)phenoxy)methyl)benzonitrile (13);
2-((3-(4-(2,6-dimethylpyridin-4-yl)-1H-pyrazol-3-yl)phenoxy)methyl)benzonitrile (14); or
4-(4-(2,6-dimethylpyridin-4-yl)-1H-pyrazol-3-yl)-2-((4-fluorobenzyl)oxy)benzonitrile (15).

15. The compound of any one of statements 1-14 wherein the compound is an inhibitor of the localization of lipoprotein CDE (LolCDE) complex of the ATP Binding Cassette transporter.

16. A pharmaceutical composition comprising a compound of any one of statements 1-15.

17. A method for treating a patient infected with Gram-negative bacteria comprising administering to the patient a therapeutically effective amount of the compound of any one of statements 1-15 or the pharmaceutical composition of statement 16, wherein the infected patient is thereby treated.

18. The method of statement 17 wherein the Gram-negative bacteria is Escherichia coli, Klebsiella pneumoniae, Enterobacter cloacae, or Salmonella typhimurium.

19. The method of statement 17 or 18 wherein the pharmaceutical composition is not antimicrobial against intestinal bacteria or commensal bacteria.

20. The method of any one of statements 17-19 wherein the pharmaceutical composition kills 50% of the Gram-negative bacteria at about 1 g/mL to about 3 g/mL.

21. The method of any one of statements 17-20 wherein the pharmaceutical composition kills 90% of the Gram-negative bacteria at about 7 g/mL to about 9 g/mL.

22. The method of any one of statements 17-21 wherein the pharmaceutical composition lowers the onset propensity of a Clostridioides difficile infection, gut dysbiosis, or a loss of intestinal symbionts.

23. The method of any one of statements 17-22 wherein the composition comprises the compound is 3-((3-(4-(2,6-dimethylpyridin-4-yl)-1H-pyrazol-3-yl)phenoxy)methyl)benzonitrile (12, lolamycin).

24. Use of the compound of any one of statements 1-15 or the pharmaceutical composition of statement 16 for the treatment of a bacterial infection, according to any one of statements 17-23.

25. The compound of any one of statements 1-15 or the pharmaceutical composition of statement 16 for preparation of a medicament for treating a bacterial infection, according to any one of statements 17-23.

### Results and Discussion

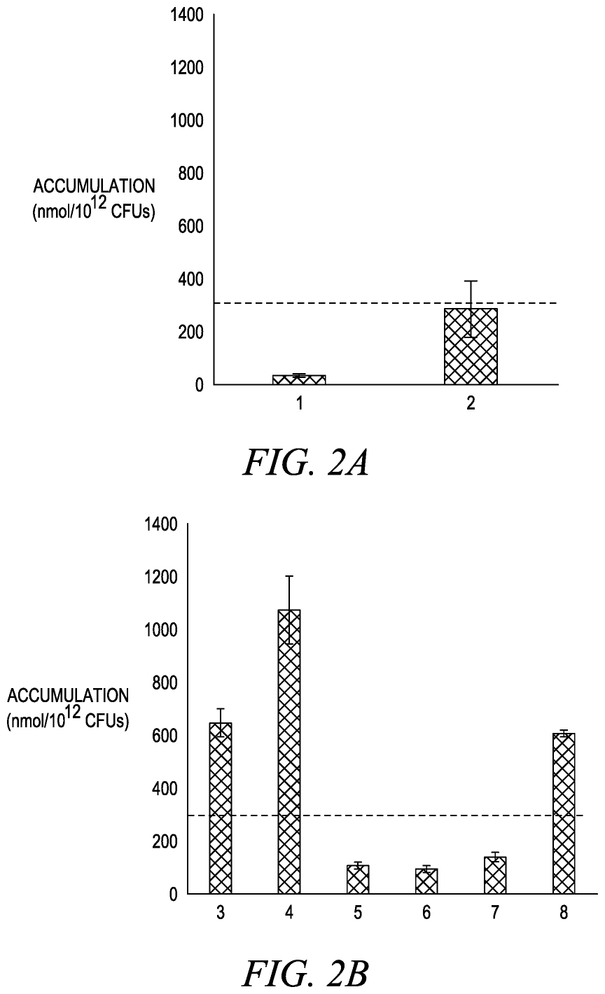

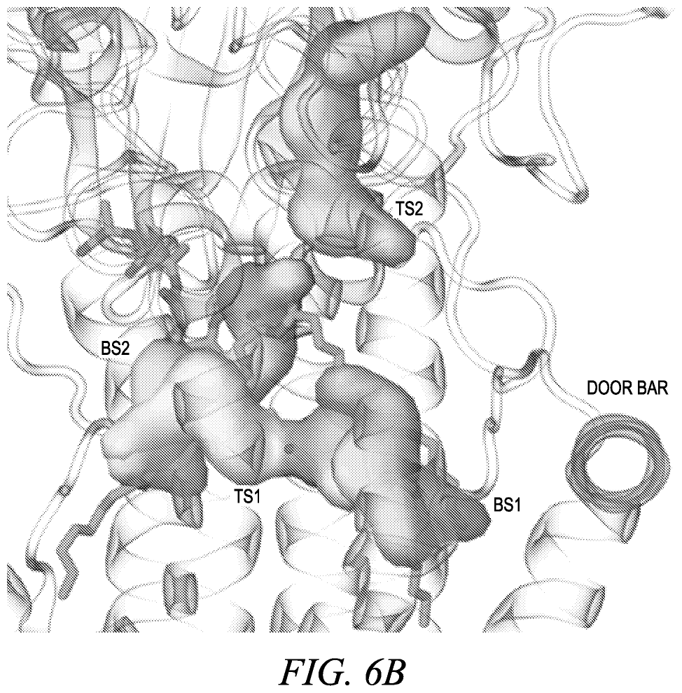

Identification of lolamycin. Screens performed at AstraZeneca in 2015 identified novel pyridine-imidazoles (J. Bacteriol. 197, 1075-1082 (2015)) and pyridine-pyrazoles (J. Bacteriol., 1726-1734 (2015)) as the first reported inhibitors of the LolCDE complex, a subcomponent of the Lol system that facilitates the release of outer membrane-specific lipoproteins (Table 1). While the lack of significant antimicrobial activity against wild-type Gram-negative pathogens (Table 1), lack of in vivo efficacy, solubility issues, and high resistance frequencies prevented these inhibitors from being pursued further, resistant mutant studies provided convincing evidence that these compounds kill bacteria through inhibition of the Lol complex. In 2018, a screen performed at Genentech revealed another inhibitor with a pyrrolopyrimidinedione core (Antimicrob. Agents Chemother. 62, (2018)) with resistant mutants mapping back to LolCDE, but again with a lack of activity against wild-type E. coli. However, compounds 1 and 2 show significant activity in efflux-deficient E. coli ΔtolC (Table 1), suggesting that if compound entry and accumulation could be facilitated, improved activity against wild-type Gram-negative strains and clinical isolates could be possible. In accordance with the previously discovered eNTRy rules, both LolCDE inhibitors meet two of the three desired parameters, possessing favorable globularities and numbers of rotatable bonds (Compound 1: globularity=0.116, rotatable bonds=3; Compound 2: globularity=0.113, rotatable bonds=5), but lacking a primary amine. Synthesis and accumulation assessment of compounds 1 and 2 confirmed that these compounds accumulate very poorly in E. coli (FIG. 2A). Therefore, improvements in whole-cell accumulation and subsequently, wild-type Gram-negative activity, through the appendage of primary amines on the parent scaffolds was sought. Overlaying the structures of compounds 1 and 2 revealed they occupy a similar conformation, and hence the methylpyridine modification of compound 1 was mapped onto compound 2 to create a hybrid scaffold.

TABLE 1
Previously reported pyridineimidazole and pyridinepyrazole LolCDE
inhibitors and their antimicrobial activities against wild-type
Gram-negative pathogens.
1

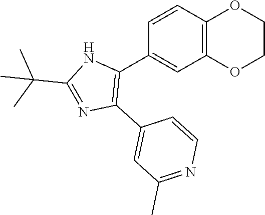 [SMILES: Cc1cc(-c2nc(C(C)(C)C)[nH]c2-c2ccc3c(c2)OCCO3)ccn1]

2

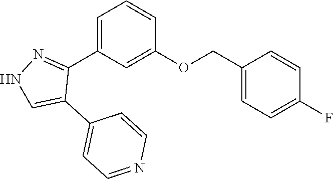 [SMILES: Fc1ccc(COc2cccc(-c3n[nH]cc3-c3ccncc3)c2)cc1]

| | MIC (μg/mL) | |
|---|---|---|
| Strain | 1 | 2 |
| E. coli ATCC 25922 | 8 | 8 |
| E. coli AR-0549 | 16 | > 32 |
| K. pneumoniae ATCC 27736 | 16 | > 32 |
| K. pneumoniae ATCC BAA-1705 | > 128 | > 32 |
| K. pneumoniae AR-0040 | 32 | > 32 |
| E. cloacae ATCC 29893 | > 128 | > 32 |
| E. cloacae AR-0163 | 64 | > 32 |
| E. cloacae BAA-2468 | 32 | > 32 |
| E. coli ΔtolC JW5503 | 0.03 | 0.06 |
| E. coli ΔrfaC JW3596 | 0.25 | 1 |

Appending amines onto this scaffold revealed that several of these compounds did have elevated whole cell accumulation, but the amine was deleterious to target engagement as indicated by a significant reduction in antibiotic activity (FIG. 2B). However, given the periplasmic nature of the Lol target, a high total whole-cell accumulation would possibly not be required for antimicrobial activity, as the periplasm represents only about 9% of the volume of the Gram-negative cell. Thus, other compounds that would be unlikely to accumulate to detectable levels in the whole-cell accumulation assay based on their structure were prepared and further assessed. This approach led to the identification of lolamycin (Table 2), a compound that now has marked activity in the laboratory strains of Gram-negative pathogens, including E. coli, K. pneumoniae, and E. cloacae (Table 2). The whole-cell accumulation of lolamycin in E. coli was found to be below the limit of detection.

TABLE 2
Antimicrobial assessment of lolamycin (12) against Gram-negative pathogens, Gram-positive pathogens,
and intestinal gut bacteria (MIC in μg/mL).
(12)

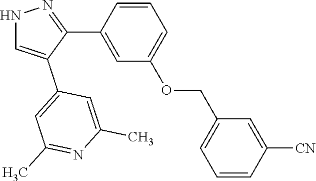 [SMILES: Cc1cc(-c2c[nH]nc2-c2cccc(OCc3cccc(C#N)c3)c2)cc(C)n1]

| Bacterial Strain | 12 | Bacterial Strain | 12 |
|---|---|---|---|
| Gram-negative permeability mutant | | | |
| Escherichia coli ΔtolC JW5503 | 0.008 | | |
| Escherichia coli ΔrfaC JW3596 | 0.06 | | |
| Wild-type Gram-negative pathogens | | Wild-type Gram-positive pathogens | |
| Escherichia coli ATCC 25922 | 1 | Staphylococcus aureus ATCC 29213 | > 128 |
| Escherichia coli AR-0349 | 1 | Bacillus subtilis PY79 | > 128 |
| Escherichia coli AR-0549 | 2 | Bacillus anthracis Sterne 7702 | > 128 |
| Salmonella typhimurium ATCC14028 | 1 | Bacillus cereus ATCC 11778 | > 128 |
| Klebsiella pneumoniae ATCC 27736 | 1 | Enterococcus faecium ATCC 19434 | > 128 |
| Klebsiella pneumoniae ATCC BAA-1705 | 4 | Enterococcus faecalis ATCC 19433 | > 128 |
| Klebsiella pneumoniae AR-0040 | 1 | Listeria monocytogenes ATCC 19115 | > 128 |
| Enterobacter cloacae ATCC 29893 | 4 | Staphylococcus epidermidis NRS101 | > 128 |
| Enterobacter cloacae ATCC BAA-2468 | 4 | Streptococcus pneumoniae Strain 1003 | > 128 |
| Enterobacter cloacae AR-0163 | 8 | Streptococcus pyogenes Strain 1055 | > 128 |
| Pseudomonas aeruginosa PAO1 | > 128 | | |
| Pseudomonas aeruginosa PAO1 Δ6 | > 128 | | |
| Acinetobacter baumannii ATCC 19606 | > 128 | | |
| Acinetobacter baumannii ATCC 19606 Δ3 | 32 | | |
| Gram-negative gut commensal bacteria | | Gram-positive gut commensal bacteria | |
| Bacteroides fragilis ATCC 25285 | > 128 | Enteroccocus faecalis KLE 2341 | > 128 |
| Bacteroides vulgatus KLE 2303 | > 128 | Streptococcus salivarius KLE 2370 | > 128 |
| Bacteroides cellulosilyticus KLE 2342 | > 128 | Streptococcus parasanguinis KLE 2375 | > 128 |
| Stenotrophomonas maltophilia ATCC 13637 | > 128 | Bifidobacterium longum ATCC BAA-999 | > 128 |
| Faecalibacterium prausnitzii ATCC 27768 | > 128 | Lactobacillus reuteri ATCC 23272 | > 128 |
| Morganella morganii ATCC 25380 | > 128 | Blautia producta ATCC 23272 | > 128 |
| Proteus mirabilis ATCC 35659 | > 128 | | |

| | |
|---|---|
| Mammalian cell lines (IC₅₀ in μM) | 85.3 ± 11.8 (HFF-1) \| 41.9 ± 0.3 (A-549) |

Assessment of lolamcyin against a range of Gram-positive and Gram-negative commensal bacteria revealed it to be inactive up to its aqueous solubility limit (128 μg/mL, Table 2). This panel of obligate anaerobes includes Bacteroides species, the most predominant Gram-negative organisms in the human gut. Lolamycin was also assessed against a panel of Gram-positive aerobic pathogenic bacteria and was also found to be inactive against these organisms (Table 2). Finally, assessment of lolamycin against wild-type strains of Gram-negative pathogens Pseudomonas aeruginosa or Acinetobacter baumannii, or the efflux-deficient strains, also revealed lolamycin to be inactive. The selectivity of lolamycin for E. coli, K pneumoniae, and E. cloacae over other pathogenic and commensal bacteria (Gram-positive and Gram-negative) is consistent with the sequence homology of the LolCDE complex, with high similarity between E. coli, K pneumoniae, and E. cloacae but low relative to the others (Table 2). Lolamycin also had minimal toxicity to mammalian cell lines (Table 2).

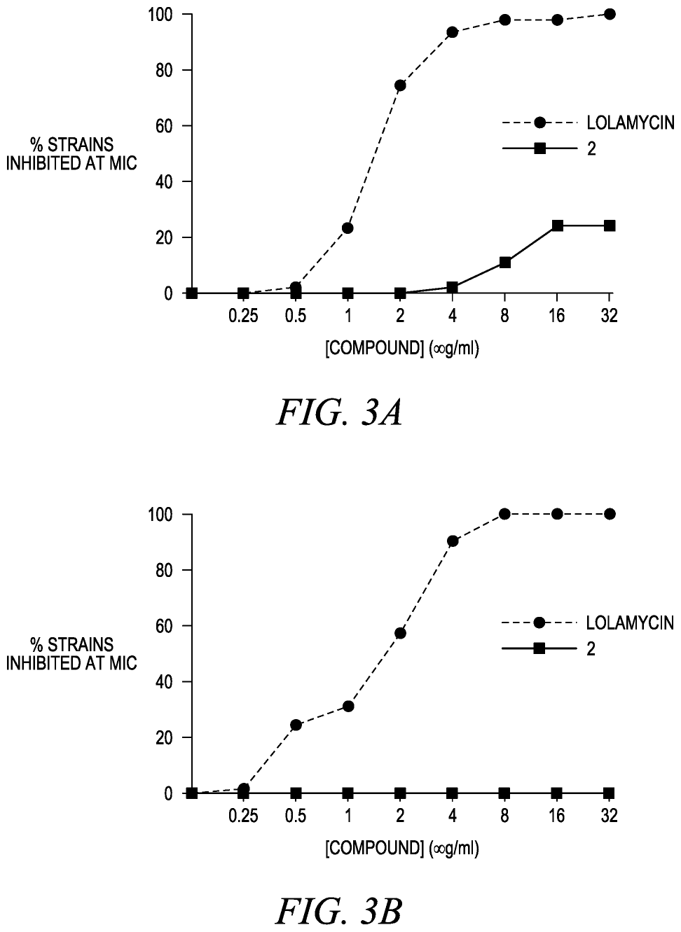

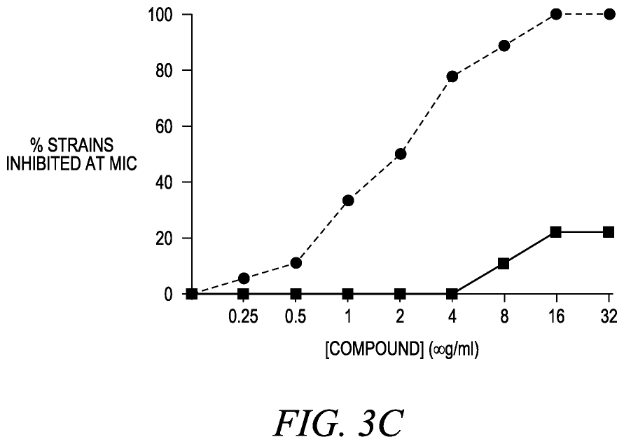

Activity of lolamycin against multidrug-resistant clinical Isolates. Lolamycin was further evaluated against an extensive panel of multidrug-resistant clinical isolates of E. coli, K. pneumoniae, and E. cloacae (FIG. 3). This collection of clinical isolates contains strains with a variety of resistance profiles, including resistance to antibiotics such as carbapenems, aminoglycosides, sulfonamides, trimethoprim, tetracyclines, and colistin (Table 8, Example 4). In these susceptibility studies lolamycin kills 50% of the strains at ˜2 μg/mL and 90% of the strains at ˜8 μg/mL (FIG. 3), and markedly outperforms progenitor compound 2, which has only minimal activity (Table 9-11, Example 4).

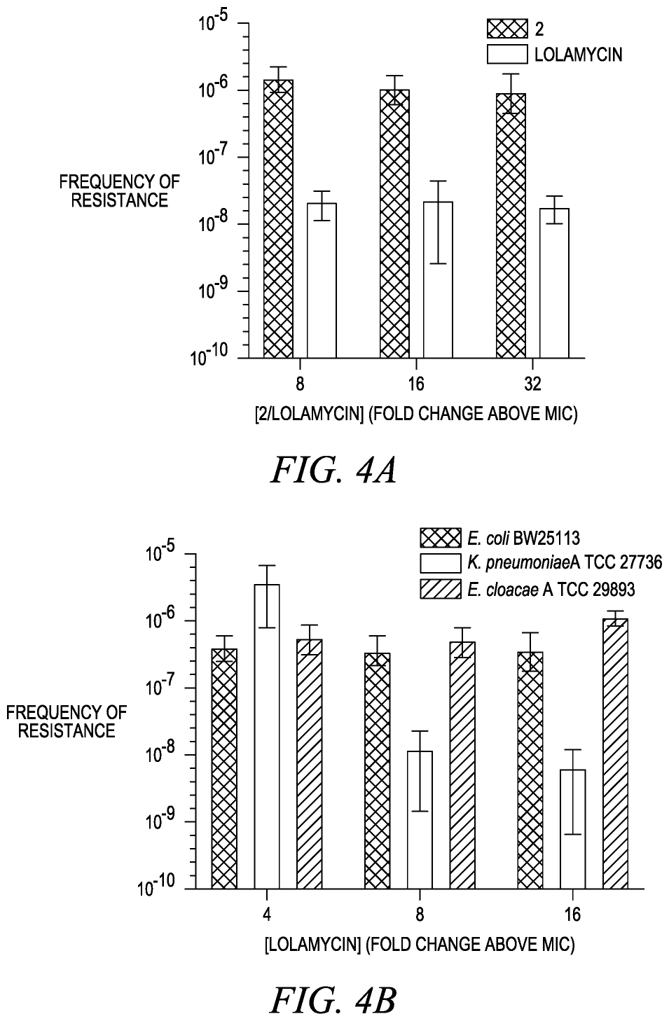

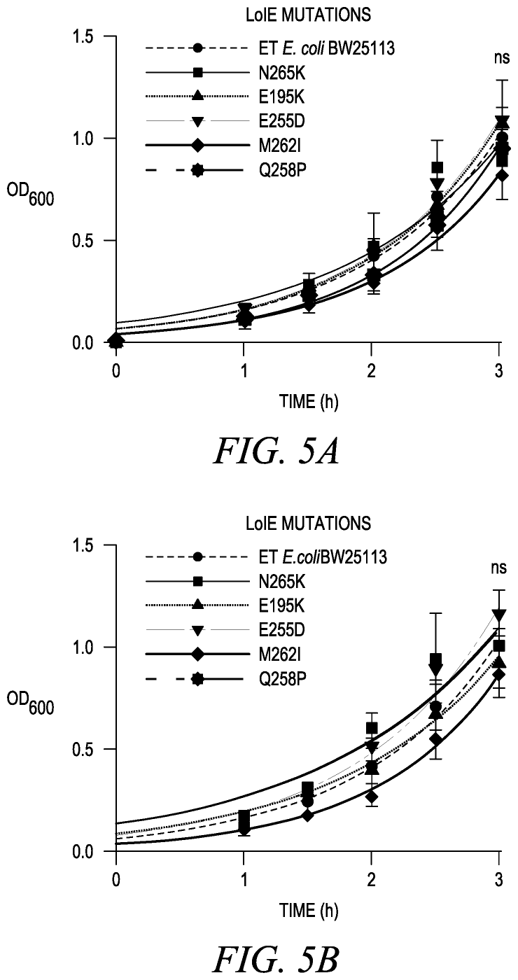

Mode of action of lolamycin. Spontaneous mutants resistant to lolamycin were generated via the large inoculum method, first in efflux pump-deficient E. coli ΔtolC (JW5503) so that lolamycin could be directly compared to compound 2. At 32×MIC in JW5503, compound 2 shows a resistance frequency of 9.2×10⁻⁷ while lolamycin shows a resistance frequency of 1.8×10⁻⁸ (FIG. 4A). Resistant mutants to lolamycin were then generated in wild-type E. coli BW25113, wild-type K. pneumoniae ATCC 27736, and wild-type E. cloacae ATCC 29893. At 8×MIC, lolamycin shows a resistance frequency of 3.4×10⁻⁷ in wild-type E. coli, 1.2×10⁻⁸ in wild-type K. pneumoniae, and 5.2×10⁻⁷ in wild-type E. cloacae (FIG. 4B). The lol locus was sequenced from 21 of these E. coli BW25113 colonies, and 9 different mutations were observed (Table 3). Resistance mutations to lolamycin mapped back to individual amino acid changes in the LolC and LolE proteins, implicating the lipoprotein transport pathway as the target of lolamycin. Changes in fitness were assessed by evaluating growth rates for lolamycin-resistant mutants and wild-type E. coli BW25113. There was no statistically significant change in growth rates for resistant mutants and wild-type E. coli (FIG. 5).

Furthermore, molecular modeling was used to characterize the binding sites of lolamycin and better understand the basis of lolamycin inhibition of LolCDE. To do this, ensemble docking was performed as an initial screening for lolamycin binding poses. Previous Cryo-EM experiments have provided multiple LolCDE conformations including a lipoprotein bound pre-transport conformation (PDB ID: 7MDX) with an outward-opened transmembrane domain (TMD) that forms a V-shaped central channel (Nature Structural & Molecular Biology 28, 347-355 (2021)). Initially, lolamycin was hypothesized to bind to the pre-transport state since it has the most accessible binding site. To probe binding to the pre-transport state, an apo structure of LolCDE was generated by removing lipoprotein from the 7MDX structure. Then, extensive equilibrium molecular dynamics (MD) simulations of apo protein in a realistic lipid bilayer were performed, generating 2.5 μs of total simulation data to exhaustively sample protein conformational space.

TABLE 3
Prevalence of LolCDE mutations in E. coli BW25113
resistant to lolamycin. Resistant colonies of E. coli BW25113
were isolated following selection at 32-fold the MIC of lolamycin.

| Fold MIC | Lol Mutant | MIC (μg/mL) | Colonies |
|---|---|---|---|
| 32 | D264N (LolE) | >128 | 2/21 |
| 32 | I206N (LolE) | >128 | 1/21 |
| 32 | N265K (LolC) | >128 | 2/21 |
| 32 | L199P (LolE) | >128 | 1/21 |
| 32 | E255D (LolC) | >128 | 2/21 |
| 32 | M262I (LolC) | >128 | 2/21 |
| 32 | E195K (LolC) | >128 | 7/21 |
| 32 | F367S (LolE) | >128 | 3/21 |
| 32 | Q258P (LolC) | >128 | 1/21 |

The free energy landscape of these simulations was projected onto representative reaction coordinates, which indicated a diversity of sampled protein conformations. Thus, to thoroughly sample possible binding sites within the protein, protein conformations were discretized using RMSD-based clustering relative to the transmembrane domain (TMD). Then, AutoDock Vina was used to dock lolamycin, obtaining 10 poses for each protein conformation. Protein-inhibitor poses contacting key residues resolved from the resistant mutant studies were identified and a representative pose with the best docking score for each protein conformation was chosen. These 10 protein-inhibitor poses were embedded in lipid bilayer and independently simulated for 100 ns each.

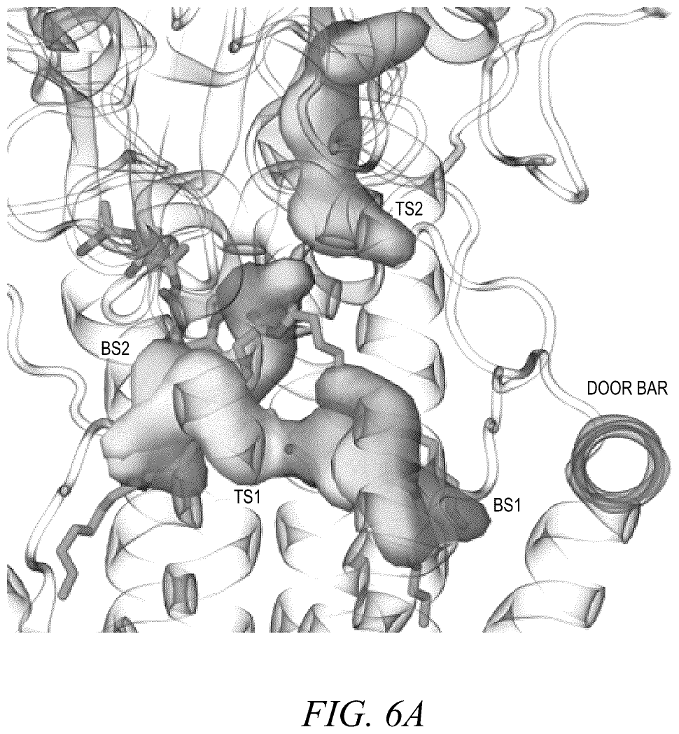

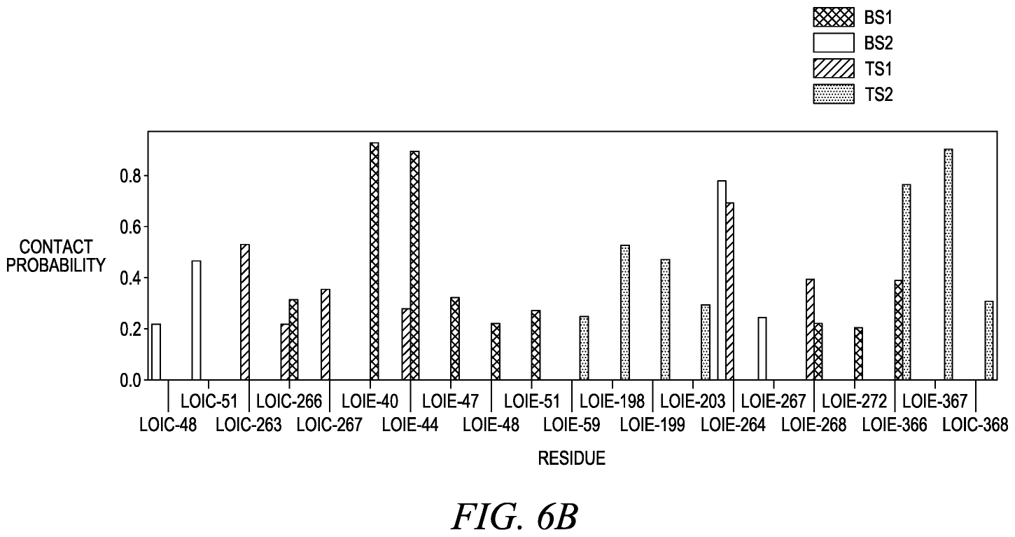

From these protein-inhibitor simulations, 4 high occupancy clusters of lolamycin binding were identified, and as such, were extended for an additional 500 ns each. From the extended simulations, the new dataset was re-clustered and conservation of the 4 major clusters was observed. Two of these clusters lie within stabler binding sites (BS1 and BS2) with greater residence time than the clusters in the transient sites (TS1 and TS2) (FIG. 6). Furthermore, lolamycin within BS1 and BS2 overlays with the Cryo-EM density of the native substrate, LPP lipoprotein and interacts with several residues that interact with lipoprotein. Point mutation of these residues has been shown to lead to deleterious effects in lipoprotein shuttling and cell viability. This suggests lolamycin competitively inhibits the lipoprotein transport pathway by physically occupying sites of lipoprotein binding.

To clarify the importance of the transient sites in relation to the binding sites, the estimated free energy from all simulations was projected onto the distance from the BS1 and BS2 cluster centers. The free energy landscape indicates a low energetic barrier (˜2.4 kcal/mol) for transition between TS1 and BS2 (FIG. 6). Furthermore, TS1 to BS2 interconversions were observed in these simulations indicating a possible role for TS1 as an intermediate state in the binding mechanism of lolamycin to BS2. Activity recovery mutations appear to interact with the inhibitor within TS1, indicating that these mutations may hinder transition into the BS2 binding site. TS2 appears to lie within a possible ingress/egress pathway for lolamycin through LolCDE. Furthermore, activity recovery mutations also line this pathway and interact with lolamycin in TS2. This suggests an important role for TS2 as an intermediate state in the ingress/egress pathway of lolamycin.

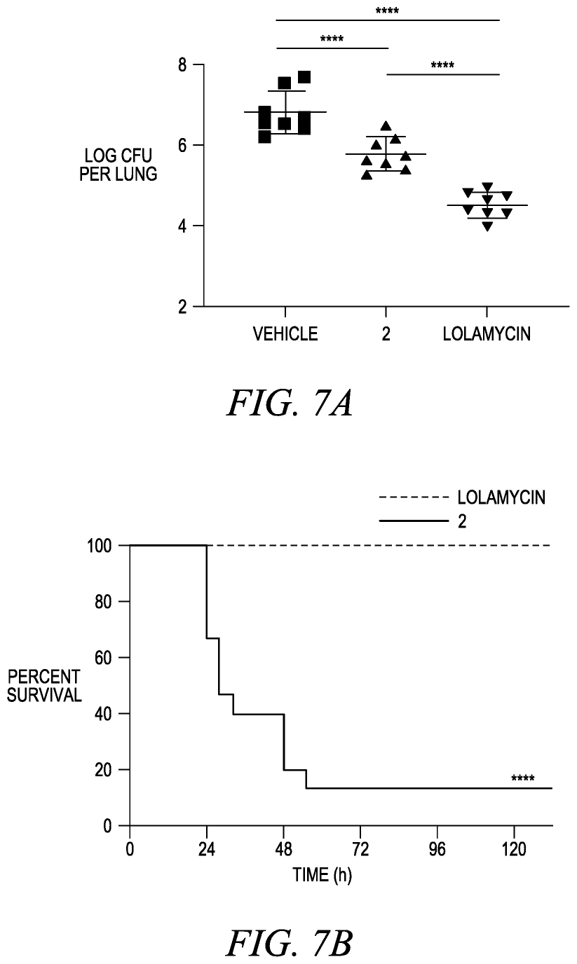

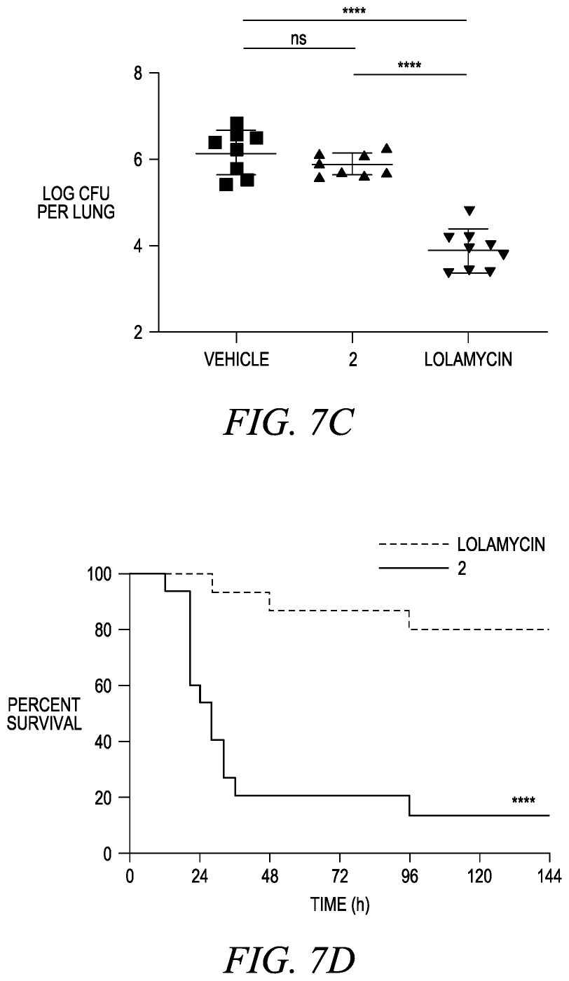

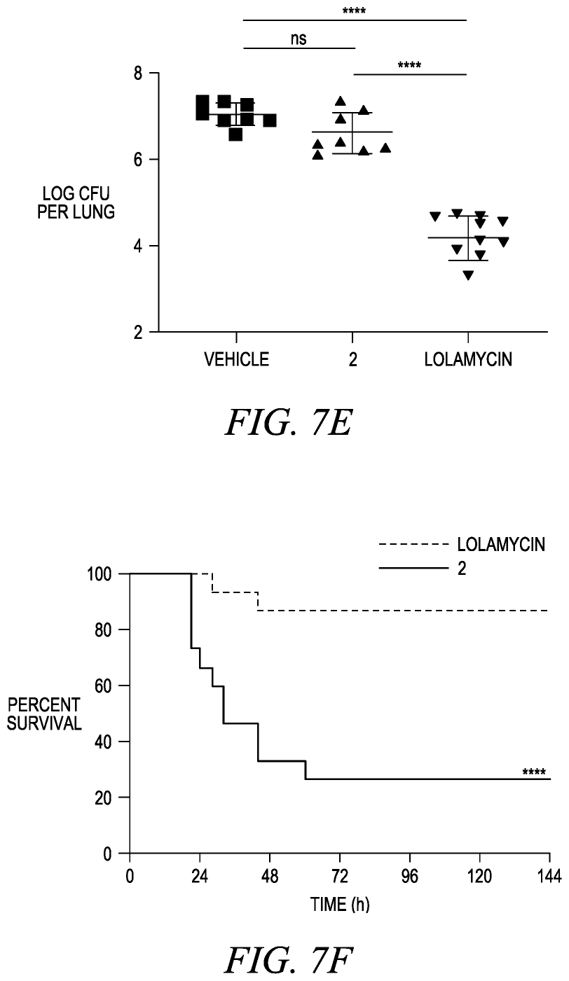

In vivo efficacy of lolamcyin. Compound 2 and lolamycin were well-tolerated by mice when administered at 100 mg/kg twice a day for three days via intraperitoneal injection (IP), so this dosing strategy was used for infection models. Compound 2 and lolamycin were evaluated head-to-head in two types of infection models: an acute pneumonia infection model examining bacterial burden and septicemia model analyzing overall survival. Mice were infected with E. coli AR-0349, a colistin-resistant clinical isolate, where lolamycin has an MIC of 1 μg/mL and compound 2 has an MIC of >32 μg/mL. Mice treated with lolamycin showed a 2-log reduction in bacterial burden (FIG. 7A). Additionally, 100% of mice burdened with septic infection were successfully rescued (FIG. 7B); compound 2 fared poorly in these experiments compared to lolamycin. Analogous experiments showed the efficacy of lolamycin against mice infected with colistin-resistant K. pneumoniae (FIG. 7C), carbapenem-resistant K. pneumoniae (FIG. 7D), and colistin-resistant E. cloacae (FIG. 7E-F).

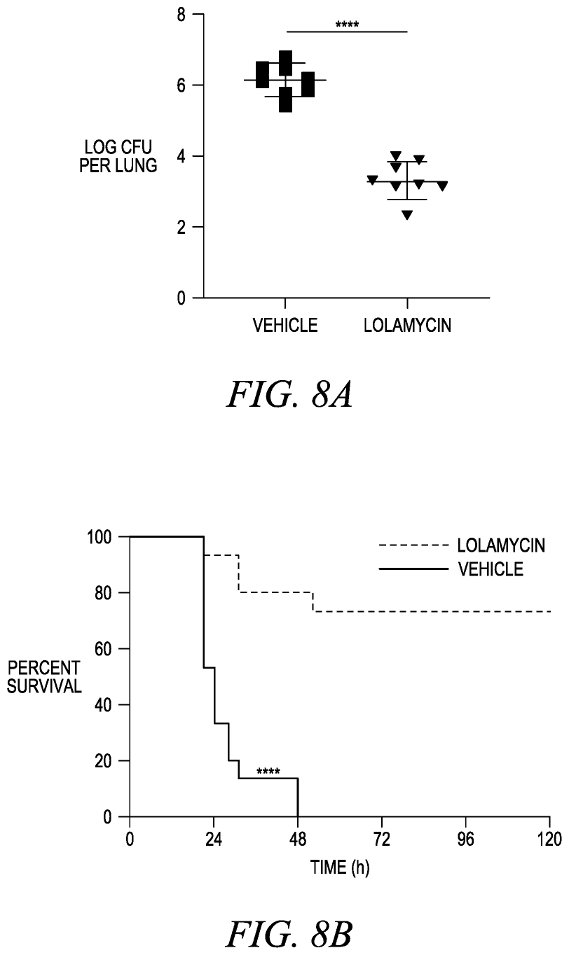

With the superiority of lolamycin relative to compound 2 obvious from these infection models, attention turned to evaluation of efficacy when given orally as a prelude to the gut microbiome studies. Pharmacokinetic experiments revealed significant oral bioavailability for lolamycin (Table 4), and lolamycin was well-tolerated when administered to mice via oral gavage at 200 mg/kg twice a day for three days, and this dosing regimen was used for infection models. Lolamycin was again evaluated in an acute pneumonia infection model and septicemia model using colistin-resistant E. coli AR-0349. Mice treated with lolamycin showed a 3-log reduction in bacterial burden when compared to treatment with vehicle (FIG. 8A). Additionally, over 70% of mice burdened with septic infection were successfully rescued (FIG. 8B).

TABLE 4
Pharmacokinetic studies of lolamycin via intraperitoneal (IP) and
oral (PO) administration.

| PK at 200 mg/kg | IP injection | Oral gavage |
|---|---|---|
| AUC (min * ng/mL) | 2,605,393 | 1,231,493 |
| Half-life (min) | 124 | 66 |
| Cmax (μM) | 38.2 | 26.4 |

$Oral Bioavailability = \frac{{AUC}_{PO} \times IP Dose}{{AUC}_{IP} \times IP Dose}$ $Oral Bioavailability = 47.3 \%$

C57BL/6 mice were treated with lolamcyin at a single dose of 200 mg/kg via IP injection (formulated in 50% DMSO and 50% PEG400) or oral gavage (formulated in 20% DMSO, 30% water, and 50% PEG400) with three mice per time point (0, 15, 30, 45, 60, 120, 240, 480, 960, and 1,440 min). Mice were then sacrificed and serum concentrations of lolamycin were determined by liquid chromatography-tandem mass spectrometry (LC-MS/MS). Oral bioavailability was calculated by comparing the concentration of lolamycin in mouse serum levels resulting from these two administration routes.

Effects of lolamycin treatment on the microbiome. Lolamycin occupies an unusual position in the antibiotic arsenal, as it displays selective antimicrobial activity for pathogenic Gram-negative bacteria, having no effect on Gram-positive bacteria nor on non-pathogenic Gram-negative bacteria in the MIC assessments. Of course, the strains tested in these experiments do not reflect the full diversity of the gut microbiome, and as such, the effects of lolamycin on the gut microbiome of mice were examined. The effects of amoxicillin and clindamycin, clinically-used antibiotics with known effects on the gut microbiome, were also evaluated as a comparison. Healthy mice were treated with either vehicle (20% DMSO, 30% water, 50% PEG400), amoxicillin (broad-spectrum antibiotic), clindamycin (Gram-positive-only antibiotic), or lolamycin (Gram-negative-only) at doses known to give antibacterial efficacy in vivo, feces were collected at days 0, 7, 10, and 31, and the microbiome composition was analyzed by full-length 16s rRNA sequencing.

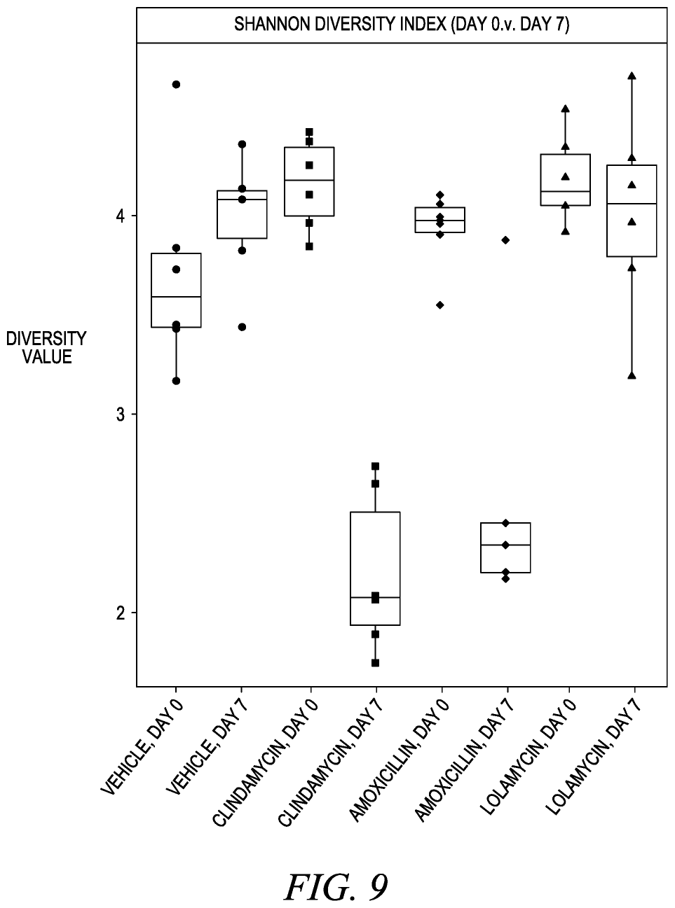

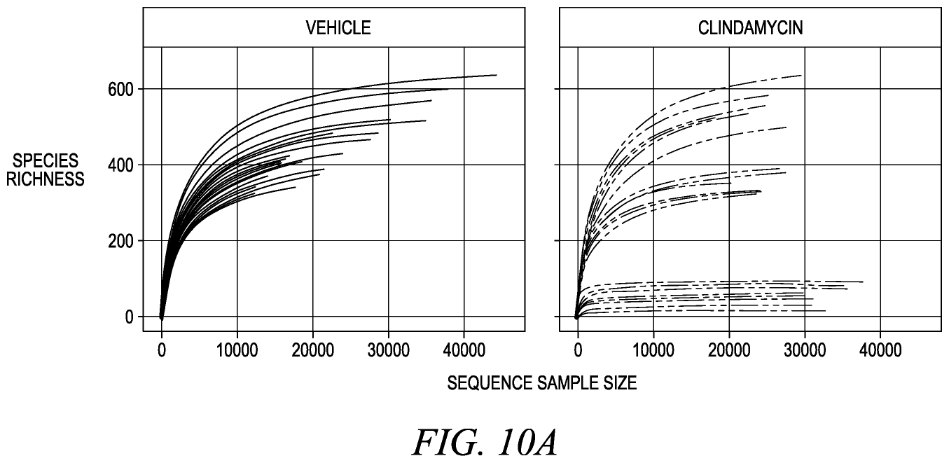

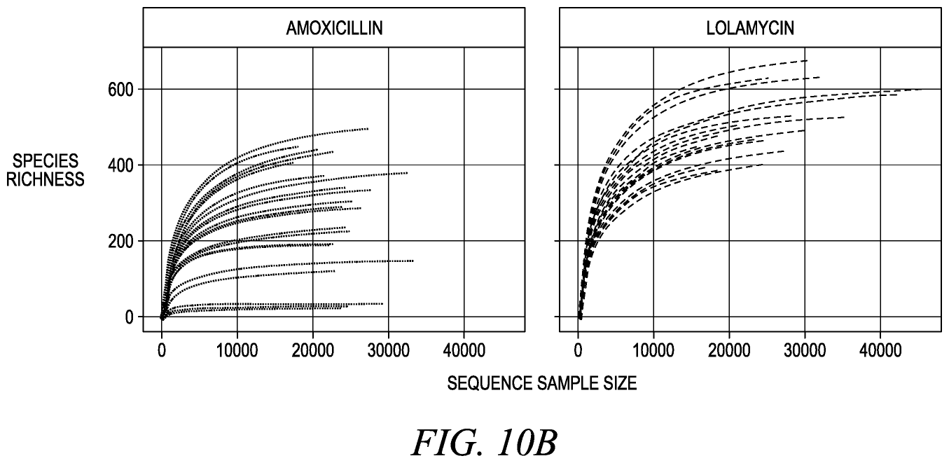

Dramatic shifts in bacterial populations were observed following administration of the antibiotic controls. Treatment with clindamycin resulted in large decreases in the members of Bacilli and Bacteroidia, with an observable increase in members of Gammaproteobacteria, detectable at four days and seven days post-treatment. Analogous shifts were also observed in mice treated with amoxicillin; Amoxicillin-treated mice showed increases in members of Bacteroidia, with subtle increases in the members of Gammaproteobacteria and decreases in Bacilli. Maximal dysbiosis was observed immediately after antibiotic treatment finished so statistical significances were determined using groups from Day 0 and Day 7. In contrast, lolamycin treatment did not cause any drastic changes in taxonomic composition over the course of the three-day treatment or the following 28-day recovery. Alpha diversity measurements including the Chao1, Simpson, and Shannon Indexes as well as rarefaction analysis were employed for examining population richness and diversity across all treatment groups (FIGS. 9 and 10).

### Pharmaceutical Formulations

The compounds described herein can be used to prepare therapeutic pharmaceutical compositions, for example, by combining the compounds with a pharmaceutically acceptable diluent, excipient, or carrier. The compounds may be added to a carrier in the form of a salt or solvate. For example, in cases where compounds are sufficiently basic or acidic to form stable nontoxic acid or base salts, administration of the compounds as salts may be appropriate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, α-ketoglutarate, and β-glycerophosphate. Suitable inorganic salts may also be formed, including hydrochloride, halide, sulfate, nitrate, bicarbonate, and carbonate salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

The compounds of the formulas described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The compounds described herein may be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds may also be combined with one or more excipients and used in the form of ingestible tablets, buccal tablets, troches, capsules, elixirs, suspensions, syrups, wafers, and the like. Such compositions and preparations typically contain at least 0.10% of active compound. The percentage of the compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, or sugar, and the like. A syrup or elixir may contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may be administered intravenously or intraperitoneally by infusion or injection. Solutions of the active compound or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound in the required amount in the appropriate solvent with various other ingredients enumerated above, as required, optionally followed by filter sterilization. In the case of sterile powders for the preparation of sterile injectable solutions, methods of preparation can include vacuum drying and freeze-drying techniques, which yield a powder of the active ingredient plus any additional desired ingredient present in the solution.

For topical administration, compounds may be applied in pure form, e.g., when they are liquids. However, it will generally be desirable to administer the active agent to the skin as a composition or formulation, for example, in combination with a dermatologically acceptable carrier, which may be a solid, a liquid, a gel, or the like.

Useful solid carriers include finely divided solids such as talc, clay, microcrystalline cellulose, silica, alumina, and the like. Useful liquid carriers include water, dimethyl sulfoxide (DMSO), alcohols, glycols, or water-alcohol/glycol blends, in which a compound can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are known to the art; for example, see U.S. Pat. No. 4,992,478 (Geria), 4,820,508 (Wortzman), 4,608,392 (Jacquet et al.), and 4,559,157 (Smith et al.). Such dermatological compositions can be used in combinations with the compounds described herein where an ingredient of such compositions can optionally be replaced by a compound described herein, or a compound described herein can be added to the composition.

Useful dosages of the compounds described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The compound can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compounds described herein can be effective antibiotics and have higher potency and/or reduced toxicity as compared to 4-(3-(3-((4-fluorobenzyl)oxy)phenyl)-1H-pyrazol-4-yl)pyridine (2). Preferably, compounds of the invention are more potent and less toxic than compound 2, and/or avoid a potential site of catabolic metabolism encountered with compound 2, i.e., have a different metabolic profile than compound 2.

The invention provides therapeutic methods of treating Gram-negative infections in a mammal, which methods involve administering to a mammal having the infection an effective amount of a compound or composition described herein. A mammal includes a primate, human, rodent, canine, feline, bovine, ovine, equine, swine, caprine, bovine and the like.

The ability of a compound of the invention to treat Gram-negative infections may be determined by using assays well known to the art. For example, the design of treatment protocols, toxicity evaluation, data analysis, quantification of bacterial cell kill, and the biological significance of the use of bacterial screens are known. In addition, the ability of a compound to treat Gram-negative infections may be determined using the tests as described herein.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods for Biological Experiments

Bacterial strains. Staphylococcus aureus ATCC 29213, Escherichia coli MG1655, Escherichia coli BAA-2340, Escherichia coli BAA-2469, Escherichia coli BAA-2471, Enterobacter cloacae BAA-2341, Enterobacter cloacae BAA-2468, Klebsiella pneumoniae BAA-1705, Klebsiella pneumoniae BAA-2342, Klebsiella pneumoniae BAA-2470, Klebsiella pneumoniae BAA-2472, Klebsiella pneumoniae BAA-2473, Enterococcus faecium ATCC 19434, Enterococcus faecalis ATCC 19433, Morganella morganii ATCC 25830, Proteus mirabilis ATCC 35659, and Salmonella typhimurium ATCC 14028 were obtained from the American Type Culture Collection (ATCC). Escherichia coli BW25113, Escherichia coli JW5503, and Escherichia coli JW3596 were obtained from the Keio Collection. Enterobacter cloacae ATCC 29893 was provided by W. van der Donk (University of Illinois at Urbana-Champaign). Bacillus cereus ATCC 11778 and Listeria monocytogenes ATCC 19115 were provided by A. Salyers (University of Illinois at Urbana-Champaign). Bacillus anthracis Sterne 7702 was provided by D. Mitchell (University of Illinois at Urbana-Champaign). Bacillus subtilis PY79 was provided by E. Garner (Harvard University). Staphylococcus epidermidis NRS 101 was obtained from the Network on Antimicrobial Resistance in S. aureus (NARSA). Streptococcus pneumoniae Strain 1003 and Streptococcus pyogenes Strain 1055 were obtained from Cubist. Streptococcus salivarius KLE 2370, Streptococcus parasanguinis KLE 2375, Bacteroides cellulosilyticus KLE 2342, Bacteroides fragilis ATCC 25285, Bacteroides vulgatus KLE 2303, Faecalibacterium prausnitzii ATCC 27768, Enterococcus faecalis KLE 2341, Bifidobacterium longum ATCC BAA-999, Lactobacillus reuteri ATCC 23272, Stenotrophomonas maltophilia ATCC 13637, and Blautia producta ATCC 27340 were provided by K. Lewis (Northeastern University). Clinical isolates of E. coli, K. pneumoniae, and E. cloacae were obtained from the Centers for Disease Control and Prevention.

Antimicrobial susceptibility assays. Susceptibility testing was performed in biological triplicate, using the micro-dilution broth method as outlined by the Clinical and Laboratory Standards Institute. Aerobic bacteria were cultured with cation-adjusted Mueller-Hinton Broth (Sigma-Aldrich; catalogue number: 90922) or Todd Hewitt Broth (Sigma-Aldrich; catalogue number: T1438) in round-bottom 96-well plates (Corning; catalogue number: 3788). Susceptibility testing with KLE collection bacteria was determined under anaerobic conditions (Coy Anaerobic chamber, 37° C., 5% H₂, 10% CO₂, 85% N₂). Overnight cultures were grown in brain-heart infusion broth (Fisher Scientific; catalogue number: DF0418-17-7) supplemented with 0.5% yeast extract, 0.1% L-cysteine hydrochloride, and 15 μg/mL hemin (BHI-ych). Human serum albumin was purchased from Sigma-Aldrich (catalogue number: A1653).

Accumulation assay. The accumulation assay (N. Engl. J. Med. 375, 2369-2379 (2016)) was performed in triplicate in batches of ten samples, with each batch containing tetracycline as a positive control. E. coli BW25113 was used in these experiments. For each replicate, 2.5 ml of an overnight culture of E. coli was diluted into 250 ml of fresh Luria Bertani (LB) broth (Lennox) and grown at 37° C. with shaking to an optical density (OD600) of 0.55. The bacteria were pelleted at 3,220 r.c.f. for 10 min at 4° C. and the supernatant was discarded. The pellets were re-suspended in 40 ml of phosphate buffered saline (PBS) and pelleted as before, and the supernatant was discarded. The pellets were re-suspended in 8.8 mL of fresh PBS and aliquoted into ten 1.5 ml Eppendorf tubes (875 μl each). The number of colony-forming units (CFUs) was determined by a calibration curve. The samples were equilibrated at 37° C. with shaking for 5 min, compound was added (final concentration=50 μM), and then samples were incubated at 37° C. with shaking for 10 min. After incubation, 800 μl of the cultures were carefully layered on 700 μl of silicone oil (9:1 AR20/Sigma High Temperature, cooled to −78° C.). Bacteria were pelleted through the oil by centrifuging at 13,000 r.c.f. for 2 min at room temperature (supernatant remains above the oil); the supernatant and oil were then removed by pipetting. To lyse the samples, each pellet was suspended in 200 μl of water, and then they were subjected to three freeze-thaw cycles of three minutes in liquid nitrogen followed by three minutes in a water bath at 65° C. The lysates were pelleted at 13,000 r.c.f. for 2 min at room temperature and the supernatant was collected (180 l). The debris was resuspended in 100 l of methanol and pelleted as before. The supernatants were removed and combined with the previous supernatants collected. Finally, the remaining debris was removed by centrifuging at 20,000 r.c.f. for 10 min at room temperature. Supernatants were analyzed by LC-MS/MS.

Samples were analyzed with the 5500 QTRAP LC/MS/MS system (AB Sciex) with a 1200 series HPLC system (Agilent Technologies) including a degasser, an autosampler, and a binary pump. The liquid chromatography separation was performed on an Agilent SB-Aq column (4.6×50 mm, 5 m) (Agilent Technologies) with mobile phase A (0.1% formic acid in water) and mobile phase B (0.1% formic acid in acetonitrile). The flow rate was 0.3 m/min. The linear gradient was as follows: 0-3 min, 100% mobile S18 phase A; 10-15 min, 2% mobile phase A; 15.5-21 min, 100% mobile phase A. The autosampler was set at 5° C. The injection volume was 15 μL. Mass spectra were acquired with both positive electrospray ionization at the ion spray voltage of 5,500 V and negative electrospray ionization at the ion spray voltage of −4,500 V. The source temperature was 450° C. The curtain gas, ion source gas 1, and ion source gas 2 were 33, 50 and 65, respectively. Multiple reaction monitoring was used to quantify metabolites. Power analysis was not used to determine the number of replicates. Error bars represent the standard error of the mean of three biological replicates. Statistical significance of accumulation was determined using a two sample Welch's t-test (one-tailed test, assuming unequal variance) relative to the negative controls. All compounds evaluated in biological assays were >95% pure.

Cell culture. HFF-1 cells (male, newborn) were obtained from the ATCC. HFF-1 cells were grown in Dulbecco's Modified Eagle's minimum essential medium with 15% fetal bovine serum (Gemini Benchmark; catalogue number: 100-106), 100 μg/mL penicillin and 100 μg/mL streptomycin. A-549 cells (male, adult) were obtained from the ATCC. A-549 cells were grown in Roswell Park Memorial Institute medium with 10% fetal bovine serum (Gemini Benchmark; catalogue number: 100-106), 100 μg/mL penicillin and 100 μg/mL streptomycin. All cells were cultured at 37° C. under a 5% CO₂ environment. Cell lines were authenticated externally by a commercial vendor and inspected visually in house. Cell lines were not tested for Mycoplasma contamination. The media was prepared by the University of Illinois School of Chemical Sciences Cell Media Facility.

Cell viability. HFF-1 cells were seeded (8,000 cells/well) in a 96-well plate (Greiner Bio-One; catalogue number: 655180) and allowed to attach overnight. Cells were treated with investigational compounds in dimethyl sulfoxide (DMSO). For half-maximum inhibitory concentration (IC₅₀) determination, the concentrations of the tested compounds were 100 nM to 1000 μM (1% DMSO final; 100 μL/well). Raptinal (100 μM) was used as a dead control. On each plate, at least three technical replicates per compound were performed. After 24 h post-treatment, cell viability was assessed using the Alamar Blue method. Stock Alamar Blue solution (10 μL 440 μM resazurin (Sigma-Aldrich; catalogue number: R7017) in sterile 1×PBS) was added to each well, and the plate was incubated for 3-4 h. Conversion of Alamar Blue was measured with a plate reader (SpectraMax M3; Molecular Devices) by fluorescence (excitation wavelength: 555 nm; emission wavelength: 585 nm; cutoff 570 nm; autogain). Percentage death was determined by normalizing to DMSO-treated cells and Raptinal-treated cells. For IC₅₀ determination, the data were plotted as compound concentration versus the percentage of dead cells and fitted to a logistic-dose-response curve using OriginPro 2015 (Origin Lab). The data were generated in triplicate, and IC₅₀ values were reported as the average of three separate experiments along with s.e.m. values.

Selection of resistant mutants. Resistant mutants were selected using the large inoculum method. Briefly, E. coli BW25113, K. pneumoniae ATCC 27736, or E. cloacae ATCC 29893 (˜1.0×10⁷ c.f.u.) was plated on 100-mm plates of Luria Bertani agar containing 8, 16, 32, or 64 μg/mL, lolamycin. For E. coli ΔtolC (JW5503), ˜1.0×10⁷ c.f.u. was plated on 100-mm plates of Luria Bertani agar containing 0.063, 0.13, 0.25, 0.5 μg/mL lolamycin. Colonies were visible after incubating at 37° C. for 48 h. Resistant colonies were confirmed by growing colonies in selective media with the same concentration of lolamycin.

Sequencing of LolCDE complex. LolC, LolD, and LolE genes were amplified by colony polymerase chain reaction (PCR). Colonies were picked and diluted in 100 μL of sterile H₂O. PCR reactions were set up by combining 25 μL MiFi Mix (Bioline, London, UK), 1 μL 20 μM primer mix (N3P-149 (5′-GGTTATGACCCGAGGTGTAATG-3′) and N3P-150 (5′-CTACCAGCTCACACACTGCATC-3′)), 10 μL template, and 14 μL H₂O. The reaction was performed on a C1000 Themal Cycler (Bio-Rad) under the following conditions for 35 cycles: initial denature: 94° C. for 5 min; denature: 94° C. for 30 s; anneal: 72° C. for 2 min. A 10 μL portion of the PCR reaction mixture was analyzed by agarose gel to confirm the product. PCR reactions were purified using GeneJET PCR Purification Kit (Thermo Scientific). PCR amplicons were submitted to the Core DNA Sequencing Facility at the University of Illinois at Urbana-Champaign for Sanger sequencing with the following primers to sequence LolC, LolD, and LolE (N3P-149, N3P-150, N3P-151, N3P-152, N3P-153, N3P-154, N3P-155, N3P-156, N3P-157). See Table 5 for the sequences of the primers used in this study. All primers were obtained from Integrated DNA technologies.

TABLE 5
Sequences of the primers for LolCDE complex.

| Name | SEQ ID | Primer Sequence | Gene(s) |
|---|---|---|---|
| N3P-149 | SEQ ID NO: 1 | GGTATGACCCGAGGTGTAAT | Forward |
| | | G | LolCDE |
| N3P-150 | SEQ ID NO: 2 | CTACCAGCTCACACACTGCA | Reverse |
| | | TC | LolCDE |
| N3P-151 | SEQ ID NO: 3 | CATGGCAATTTAGCCGATTG | Forward |
| | | | LolC |
| N3P-152 | SEQ ID NO: 4 | GAAACAAACTGACCTCGAGC | Forward |
| | | | LolC |
| N3P-153 | SEQ ID NO: 5 | CATGATGGTCTTTATGGTGC | Forward |
| | | | LolC |
| N3P-154 | SEQ ID NO: 6 | CAGTTTCACCACCTGCTGC | Forward |
| | | | LolD |
| N3P-155 | SEQ ID NO: 7 | GGTATTGATCGTCGGCTTAA | Forward |
| | | G | LolE |
| N3P-156 | SEQ ID NO: 8 | CAATATCTTGATATGGGTTC | Forward |
| | | | LolE |
| N3P-157 | SEQ ID NO: 9 | CAACTGCCGACCGCTATC | Reverse |
| | | | LolE |

Equilibrium apo simulations. Initial systems were built using a lipoprotein-bound Cryo-EM structure of LolCDE (PDB ID: 7MDX). Then the lipoprotein was removed resulting in an apo structure for simulation. The apo protein was then embedded into a symmetric membrane approximating the E. coli inner membrane with the following lipid abundances: 12.7% POPE, 12.7% QMPE, 43.1% PMPE, 7.8% OYPE, 9.8% PMPG, 8.8% PYPG, 4.9% PVCL2.

The protein-membrane system was then solvated with TIP3P water and buffered in 0.15 M NaCl.

Each step of the membrane building process was carried out using the Membrane Builder module of CHARMM-GUI. The lipid environment was then shuffled using Membrane Mixer plugin of VMD to generate five systems with varying lipid arrangements. Each system was slowly equilibrated with 1 kcal/mol restraints on lipid headgroups and protein heavy atoms over a 2 ns period in the NVT ensemble. After equilibration each replica was simulated for 500 ns in NPT.

To identify distinct protein conformations, RMSD based clustering of the transmembrane region of LolCDE was performed on the 2.5 μs data set using the VMD Cluster utility (3 Å cutoff). This yielded 10 distinct conformations of apo LolCDE. Lolamycin was docked to each of the protein conformations using AutoDock Vina.

Inhibitor bound simulations. The top drug-protein pose in which the drug contacts key experimentally identified residues was chosen for additional simulation in each of the 10 protein conformations. Each lolamycin-protein system was equilibrated with 1 kcal/mol restraints on lipid headgroups, protein heavy atoms, and inhibitor over a 2 ns period in the NVT ensemble. Then the 10 systems were independently simulated for 100 ns with no restraints for a total of 1 μs of sampling.

To identify distinct protein-drug conformations, RMSD based clustering of the drug was performed on the full dataset using the VMD Cluster utility (2.75 Å cutoff) to generate 10 clusters. From the clustering two putative binding sites and two transiently bound states of lolamycin were highlighted. Each bound state was simulated for an additional 500 ns for further sampling.

All MD simulations were performed using NAMD. The force field basis for all simulations was the CHARMM36 force field. Timestep was set to 2 fs. A 12-A cutoff for short-range, non-bonded interactions, with a switching starting at 10 Å was used. All long-range electrostatic interactions were calculated with the particle mesh Ewald (PME) method with grid density of 1 ∈⁻³, and PME interpolation of 6. The SHAKE algorithm was used to maintain rigidity for all bonds with hydrogen atoms. Langevin thermostat with damping coefficient of 1.0 ps⁻¹ was used to set temperature to 310 K. Nose-Hoover Langevin piston barostat with period 50 fs, and decay 25 fs) was used to maintain pressure at 1 atm. All simulations were run using flexible cell allowing dimensions of the periodic cell to independently change while maintaining x-y plane aspect ratio. Lennard-Jones and PME forces are updated every timestep.

Mouse maximum tolerated dose (MTD) of compound 2 and lolamycin. The protocol was approved by the Institutional Animal Care and Use Committee (IACUC) at the University of Illinois at Urbana-Champaign. In these studies, 10- to 12-week-old female C₅₇BL/6 mice purchased from Charles River Laboratories were used. The maximum tolerated dose (MTD) of a single compound via IP injection was determined first. Compound 2 and lolamycin were formulated in 50% DMSO, 50% PEG400. Compound 2 and lolamycin were given by intraperitoneal (IP) injection. All the mice were monitored for signs of toxicity for 2 weeks. For multiple doses, the compound was given by daily IP injection for 5 consecutive days and the mice were monitored for signs of toxicity for 1 month. The MTD was the highest dosage with acceptable toxicity (for example, <20% weight loss). Compound 2 and lolamycin were well tolerated as a single dose of 200 mg/kg. Further analysis showed that compound 2 and lolamycin were well tolerated with daily dosing of 200 mg/kg for 5 consecutive days.

Next, the MTD of lolamycin via oral gavage was determined. Lolamycin was formulated in 20% DMSO, 30% Water, and 50% PEG400. Lolamycin was given via oral gavage. All the mice were monitored for signs of toxicity for 2 weeks. For multiple doses, compound was given twice daily orally for 3 consecutive days and the mice were monitored for signs of toxicity. The MTD was the highest dosage with acceptable toxicity (for example, <20% weight loss). Lolamycin at 200 mg/kg was well tolerated twice a day for 3 days. The MTD of lolamycin was used to inform the dosing schedule used in subsequent efficacy studies.

Pharmacokinetic Assessment of compound 2 and lolamycin. The protocol was approved by the Institutional Animal Care and Use Committee (IACUC) at the University of Illinois at Urbana-Champaign. In these studies, 10- to 12-week-old female C57BL/6 mice purchased from Charles River Laboratories were used. Mice were randomly chosen and divided into subsequent groups. No additional randomization was used to allocate the experimental groups; blinding was not performed for subsequent quantitation. First, the pharmacokinetic assessment via intraperitoneal injection was determined. Compound 2 and lolamycin were formulated in 50% DMSO and 50% PEG400. Mice were treated with compound 2 (200 mg/kg) or lolamycin (200 mg/kg) via intraperitoneal injection, with three mice per time point (0, 15, 30, 45, 60, 120, 240, 480, 960, and 1,440 min). At the specified time points, mice were killed, blood was collected and centrifuged, and the serum was frozen at −80° C. until analysis. The proteins in a 10-μL aliquot of serum were precipitated by the addition of 50 μL methanol with the addition of 10 μL internal standard. The sample was then vortexed and centrifuged to remove the proteins. Supernatants were analyzed with the QTRAP 5500 LC/MS/MS system (Sciex) in the Metabolomics Laboratory of the Roy J. Carver Biotechnology Center, University of Illinois at Urbana-Champaign.

Next, the pharmacokinetic assessment via oral gavage was determined. Lolamycin was formulated in 20% DMSO, 30% water, and 50% PEG400. Mice were treated with lolamycin (200 mg/kg) via oral gavage, with three mice per time point (0, 15, 30, 45, 60, 120, 240, 480, 960, and 1,440 min). At the specified time points, mice were killed, blood was collected and centrifuged, and the serum was frozen at −80° C. until analysis similar to before. Software Analyst 1.6.2 was used for data acquisition and analysis. The 1200 Series HPLC system (Agilent Technologies) includes a degasser, an autosampler and a binary pump. The liquid chromatography separation was performed on an Agilent Zorbax SB-Aq column (4.6×50 mm; 5 μm) with mobile phase A (0.1% formic acid in water) and mobile phase B (0.1% formic acid in acetonitrile). The autosampler was set at 10° C. The injection volume was 5 μL. Mass spectra were acquired using electrospray ionization with a voltage of 5,500 V. The source temperature was 450° C. The curtain gas, ion source gas 1 and ion source gas 2 were 33, 65, and 60 μsi, respectively. Multiple reaction monitoring was used for quantitation with external standard. The limit of quantitation of (S/N=10) was 1 nM. Pharmacokinetic parameters were calculated with a one-compartment model using a nonlinear regression program (Phoenix WinNonlin Version 8.1; Certara USA).

Acute pneumonia bacterial burden model. The protocol was approved by the Institutional Animal Care and Use Committee (IACUC) at the University of Illinois at Urbana-Champaign. In these studies, 6- to 8-week-old male CD-1 mice purchased from Charles River Laboratories were used. Mice were randomly chosen and divided into subsequent groups. No additional randomization was used to allocate the experimental groups; blinding was not performed for subsequent quantitation. First, efficacy was determined by treatment of lolamycin via IP injection. Acclimated CD-1 mice were infected via intranasal inoculation of bacteria: E. coli AR0349 at 4.2×10⁸ CFU/mouse, K. pneumoniae AR0040 at 8×10⁷ CFU/mouse, or E. cloacae AR0163 at 7.2×10⁸ CFU/mouse. Infected mice were treated twice a day for 3 days with either vehicle, compound 2, or Lolamycin (intraperitoneal; 100 mg/kg). Drugs were formulated in 50% DMSO/50% PEG400 from solid immediately before treatment. At 72 h post-infection, CFU values were determined in the lungs through serial dilutions.

Next, efficacy was determined by treatment of lolamycin via oral gavage. Acclimated CD-1 mice were infected via intranasal inoculation of bacteria: E. coli AR0349 at 2.9×10⁸ CFU/mouse. Infected mice were treated twice a day for 3 days with either vehicle or lolamycin (oral gavage; 200 mg/kg). Drugs was formulated in 20% DMSO, 30% water, and 50% PEG400 from solid immediately before treatment. At 72 h post-infection, CFU values were determined in the lungs through serial dilutions. Statistical significance was determined by one-way analysis of variance (ANOVA) with Tukey's multiple comparisons test.

Bacterial sepsis survival model. The protocol was approved by the Institutional Animal Care and Use Committee (IACUC) at the University of Illinois at Urbana-Champaign. In these studies, 6- to 8-week-old male CD-1 mice purchased from Charles River Laboratories were used. Mice were randomly chosen and divided into subsequent groups. No additional randomization was used to allocate the experimental groups; blinding was not performed for subsequent quantitation. For the preparation of each inoculum, overnight cultures of clinical isolates were diluted in Luria Bertani broth and grown to log-phase growth at 37° C. First, efficacy was determined by treatment of lolamycin via IP injection. Infection was established via a 100-μL retro-orbital injection of bacteria: E. coli AR0349 at 5.2×10⁸ CFU/mouse, K. pneumoniae BAA-1705 at 5.8×10⁷ CFU/mouse, or E. cloacae AR0163 at 8.95×10⁸ CFU/mouse. Mice infected with K. pneumoniae BAA-1705 were treated once a day for 3 days with either compound 2 or Lolamycin (IP; 200 mg/kg). Mice infected with E. coli AR0349 or E. cloacae AR0163 were treated twice a day for 3 days with either compound 2 or lolamycin (IP; 100 mg/kg). Compound 2 was formulated in 50% DMSO/50% PEG400 and lolamycin was formulated in 50% DMSO/50% PEG400 or 20% DMSO/30% Water/50% PEG400 from solid immediately before treatment. For survival analyses, a Kaplan-Meier log-rank survival test was performed using GraphPad Prism.

Next, efficacy was determined by treatment of lolamycin via oral gavage. Infection was established via a 100-μL retro-orbital injection of bacteria: E. coli AR0349 at 6.0×10⁸ CFU/mouse. Mice infected with E. coli AR0349 were treated twice a day for 3 days with either vehicle or Lolamycin (oral gavage; 200 mg/kg). Lolamycin was formulated in 20% DMSO, 30% water, and 50% PEG400 from solid immediately before treatment.

Impact of lolamycin on murine fecal microbiome. The protocol was approved by the Institutional Animal Care and Use Committee (IACUC) at the University of Illinois at Urbana-Champaign. In these studies, 4-week-old male CD-1 mice purchased from Charles River Laboratories were used. Mice were randomly chosen and divided into subsequent groups. No additional randomization was used to allocate the experimental groups; blinding was not performed for subsequent quantitation. Animals were dosed twice a day for three days with amoxicillin (100 mg/kg) (GoldBio), clindamycin (100 mg/kg) (GoldBio), or lolamycin (200 mg/kg) via oral gavage, or were treated with vehicle (20% DMSO, 30% water, 50% PEG400) (n=6 per group). Fecal pellets were collected right before treatment (Day 0), four days after treatment (Day 7), seven days after treatment (Day 10), and 28 days after treatment ended (Day 31). DNA was isolated from stool pellets using the QIAamp DNA Stool Mini Kit. Sequencing of a stool pellet from each mouse before and after treatment was performed by PacBio. 16S amplicons were generated and converted to a PacBio library with the SMRTBell Express Template Prep kit 3.0. The library was sequenced on 1 SMRTcell 8M on a PacBio Sequel IIe using the CCS sequencing mode and a 30 hs movie time. CCS analysis was done using SMRTLink V 11.0 using the following parameters: ccs-min-passes 3-min-rq 0.999.

### Example 2. Materials and General Methods for Chemical Synthesis

All reactions were carried out under an atmosphere of N₂ using oven or flame dried glassware and standard syringe/septa techniques. Tert-butyl imidazole was purchased from PharmaBlock Sciences (Nanjing), Inc. Other chemical reagents were purchased from commercial sources and used without further purification. Anhydrous solvents were either purchased from commercial suppliers or dried after being passed through columns packed with activated alumina under positive pressure of nitrogen using a PureSolv MD-5 (Inert) solvent purification system. Silica gel flash column chromatography was performed on 230-430 mesh silica gel. Analytical thin layer chromatography was performed with precoated, glass-baked plates (250) and visualized by fluorescence or charring with potassium permanganate stain. The synthesis of compound 1 was performed using a previously described synthesis. (J. Bacteriol. 197, 1075-1082 (2015)) The synthesis of compound 2 was performed using a modified sequence of a previously reported synthesis. (J. Bacteriol., 1726-1734 (2015)) ¹H NMR and ¹³C NMR experiments for prepared intermediates and products were recorded on a Bruker Advance III HD 500 MHz NMR system equipped with a CryoProbe or a Varian 500 MHz spectrometer. All NMR experiments were performed at room temperature. Spectra were obtained in the following solvents (reference peaks also included for ¹H and ¹³C NMRs): CDCl₃ (¹H NMR: 7.26 ppm; ¹³C NMR: 77.20 ppm), (CD₃)₂SO (¹H NMR: 2.50 ppm; ¹³C NMR: 39.52 ppm), or CD₃OD (¹H NMR: 3.31 ppm; ¹³C NMR: 49.10 ppm). Data are presented as follows: chemical shift (ppm), multiplicity (s=singlet, d=doublet, t=triplet, dd=doublet of doublets, m=multiplet, br=broad), coupling constant J (Hz) and integration. High resolution mass spectra (HRMS) were obtained in the School of Chemical Sciences Mass Spectrometry Laboratory on a Waters Q-TOF Ultima quadrupole time of flight spectrometer using electrospray ionization ESI.

Final compounds for biological assays were purified using a Teledyne ISCO ACCQPrep HP125 Preparative HPLC (Column: Teledyne ISCO RediSep Prep C18—20 mm×250 mm, 100 Å, 5-micron). Purity of final compounds assessed for biological activity were purified to >95% as assessed by an Agilent 6230 LC/MS TOF (Column: Agilent ZORBAX Eclipse Plus C18 Rapid Resolution HT 2.1×100 mm, 1.8-micron) and/or and Agilent Technologies 1290 Infinity II UHPLC equipped with a Phenomenex Kinetex column (2.1 mm ID×50 mm, 1.7-micron particle size, 100 Å pore size).

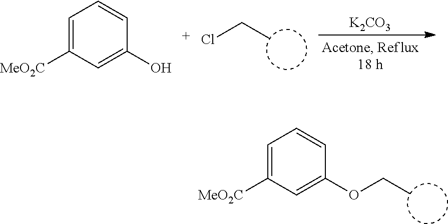

General procedure I. Methyl 3-hydroxybenzoate (1 equiv.) was dissolved in acetone (0.12M). K₂CO₃ (2.5 equiv.) and chloromethyl benzyl starting material (1.2 equiv.) were added at 55° C. The reaction was allowed to stir overnight. Reaction mixture was filtered and concentrated down. The resulting oil was triturated with 1-2 L of hexanes to afford the products (16, 17, 18, 19) as a white solid in 70-80% yield.

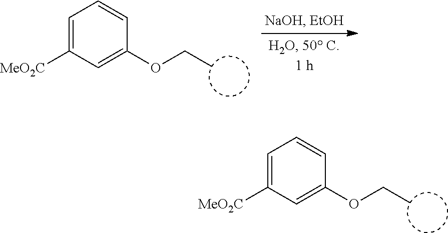

General procedure II. Benzoate starting material (1 equiv.) was suspended in EtOH (0.36M) and water (1.48M). NaOH (3.2 equiv.) was added to this suspension and the reaction was allowed to stir at 50° C. for 1 h. Reaction mixture was concentrated and the resultant product was dissolved in minimal water. The solution was made acidic by slow addition of concentrated HCl. The solid was collected by vacuum filtration and washed with water to afford the product (20, 21, 22, 23) as a white solid in 90-95% yield.

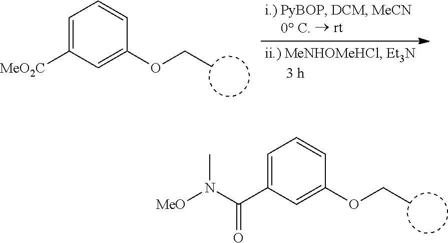

General procedure III. Benzoic acid starting material (1 equiv.) was suspended in DCM (0.58M) and ACN (0.58M) at 0° C. PyBOP was added to this suspension and allowed stir for 1 h. Triethylamine and N,O-dimethyl hydroxylamine hydrochloride were added at room temperature and the reaction was allowed to stir for 3 h. Reaction mixture was concentrated down and the resulting oil was partitioned between 1 M NaOH and CHCl₃. Layers were separated and the organic layer was concentrated down. The resulting pale yellow oil was purified via column chromatography (1:1 Hexanes/Ethyl acetate) to afford the product (24, 25, 26, 27) as a pale-yellow oil in 60-85% yield.

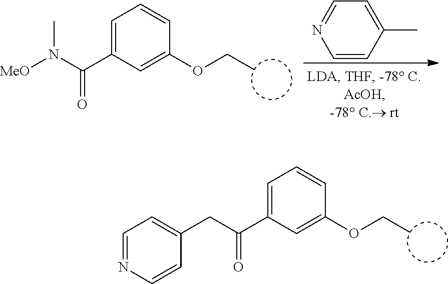

General procedure IV. 4-Methylpyridine (3 equiv.) was suspended in THF (0.99M) at 0° C. A solution of LDA (2.0M in THF) was added slowly and the reaction was stirred for 15 min. One equivalent of this methylpyridine anion solution was slowly added to a solution of N-methylbenzamide starting material (1 equiv.) in THF (0.2M) at −78° C. Reaction was stirred for 1 h followed by one more addition of the anion solution. Reaction was stirred for another hour. Reaction mixture was quenched with AcOH and warmed up to room temperature. The pH was adjusted to ˜8 with saturated aqueous NaHCO₃. Mixture was extracted three times with EtOAc. Combined organic layers were dried with MgSO₄, concentrated down, and purified via column chromatography (3:1 Hexanes/EtOAc→1:1 Hexanes/EtOAc) to afford the product (29, 32) as a yellow oil in 50-70% yield.

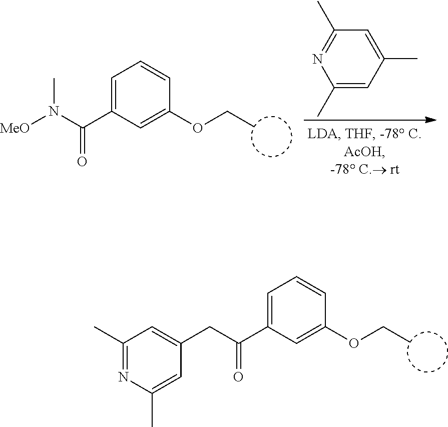

General procedure V. 2,4,6-Collidine (3 equiv.) was suspended in THF (0.99M) at 0° C. A solution of LDA (2.0M in THF) was added slowly and the reaction was stirred for 15 min. One equivalent of this trimethylpyridine anion solution was slowly added to a solution of N-methylbenzamide starting material (1 equiv.) in THF (0.2M) at −78° C. Reaction was stirred for 1 h followed by one more addition of the anion solution. Reaction was stirred for another hour. Reaction mixture was quenched with AcOH and warmed up to room temperature. The pH was adjusted to ˜8 with saturated aqueous NaHCO₃. Mixture was extracted three times with EtOAc. Combined organic layers were dried with MgSO₄, concentrated down, and purified via column chromatography (3:1 Hexanes/EtOAc→1:1 Hexanes/EtOAc) to afford the product (28, 30, 31, 33, 34) as a yellow oil in 40-60% yield.

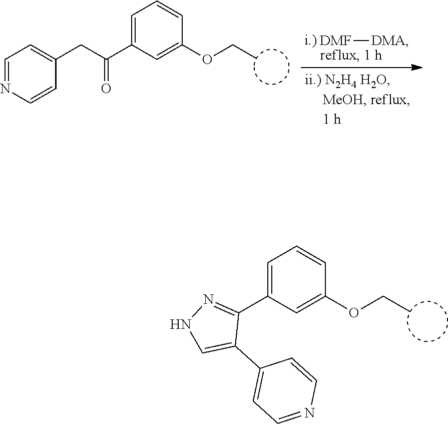

General procedure VI. Pyridine starting material was dissolved in DMF-DMA (6.5 equiv.) and the reaction was heated for 1 h under reflux. Reaction mixture was concentrated and the resulting yellow oil was dissolved in MeOH (0.35M). Hydrazine monohydrate (1.5 equiv.) was added and was heated under reflux for 1 h. Reaction material was concentrated under reduced pressure and was purified via column chromatography (3:1 Hexanes/EtOAc→1:1 Hexanes/EtOAc) to afford the final product (2, 10, 13) as a pale-yellow solid in 55-80% yield.

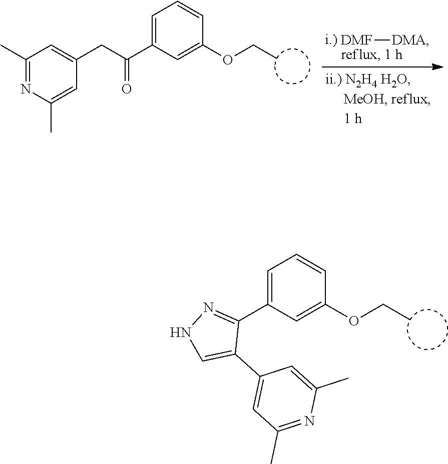

General procedure VII Dimethylpyridine starting material was dissolved in DMF-DMA (6.5 equiv.) and the reaction was heated for 1 h under reflux. Reaction mixture was concentrated and the resulting yellow oil was dissolved in MeOH (0.35M). Hydrazine monohydrate (1.5 equiv.) was added and was heated under reflux for 1 h. Reaction material was concentrated under reduced pressure and was purified via column chromatography (3:1 Hexanes/EtOAc→1:1 Hexanes/EtOAc) to afford the final product (9, 11, 12, 14, 15) as a pale-yellow solid in 55-80% yield.

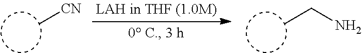

General procedure VIII. Lithium aluminum hydride in THF (1.0M) was prepared and brought down to 0° C. Benzonitrile starting material (1 equiv.) was dissolved in THF (0.2M), followed by dropwise addition to the LAH in THF solution. Reaction was allowed to stir for 3 h. Reaction material was quenched with water, 10% NaOH solution, then more water. Reaction mixture was filtered and the rinsed with MeOH. Reaction mixture was concentrated under reduced pressure and purified via column chromatography (3:1 Hexanes/EtOAc→EtOAC) to afford the product (3, 4, 5, 6, 7, 8) as a white solid in 45-75% yield.

### Example 3. Synthetic Procedures and Characterization of Compounds and Intermediates

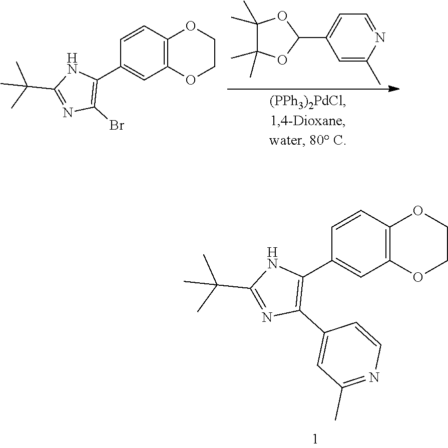

Synthetic Procedure I. In an oven-dried 4 mL vial, 4-bromo-2-(tert-butyl)-5-(2,3-dihydrobenzo[b][1,4]dioxin-6-yl)-1H-imidazole, (1 eq.) and 4,4,5,5-tetramethyl-1,3,2-dioxaborolan-2-yl)pyridine (1.5 eq.) were added to a solution of 1,4-dioxane (0.25M) and water (0.6M) that was saturated with sodium bicarbonate (base source). To this mixture, bis(triphenylphosphine) palladium (II) dichloride (0.04 mmol) was added and this was heated to 80° C. under a nitrogen atmosphere. The reaction was allowed to stir overnight. Once the reaction was complete, the crude reaction mixture was filtered. Brine was added to the filtrate, and the filtrate was extracted three times with ethyl acetate. The combined organic layers were dried over magnesium sulfate and concentrated in vacuo. The crude product was purified by silica gel chromatography (9:1 dichloromethane:methanol) to afford the desired product as colorless oil. The oil was further dissolved in a 1:1 mixture of methanol:dimethyl sulfoxide and purified further by preparative reverse phase high-performance liquid chromatography. The product fractions were collected and lyophilized to afford the product as a white powder.

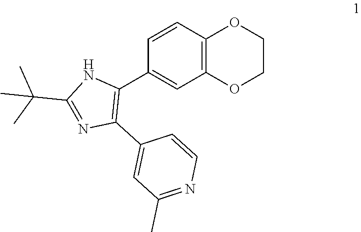 [SMILES: Cc1cc(-c2nc(C(C)(C)C)[nH]c2-c2ccc3c(c2)OCCO3)ccn1]

Compound 1, 4-(2-(tert-butyl)-5-(2,3-dihydrobenzo[b][1,4]dioxin-6-yl)-1H-imidazol-4-yl)-2-methylpyridine, was synthesized according to synthetic procedure I. The reaction was performed on a 0.53 mmol scale, providing the desired compound in 7% yield (13 mg). Experimental procedure and data for compound 1 have been published (J. Bacteriol. 197, 1075-1082 (2015)).

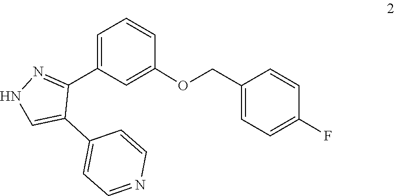 [SMILES: Fc1ccc(COc2cccc(-c3n[nH]cc3-c3ccncc3)c2)cc1]

Compound 2, 4-(3-(3-((4-fluorobenzyl)oxy)phenyl)-1H-pyrazol-4-yl)pyridine, was synthesized according to general procedure VI. The reaction was performed on a 0.17 mmol scale, providing the desired compound in 75% yield (44 mg). Compound 2 was made as previously reported (J. Bacteriol., 1726-1734 (2015)).

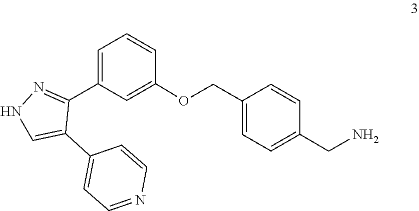 [SMILES: NCc1ccc(COc2cccc(-c3n[nH]cc3-c3ccncc3)c2)cc1]

Compound 3, (4-((3-(4-(pyridin-4-yl)-1H-pyrazol-3-yl)phenoxy)methyl)phenyl)methanamine was synthesized according to general procedure VIII. This reaction was performed on a 0.16 mmol scale, providing the desired compound in 54% yield (178 mg). HRMS (ESI): m/z calc for C₂₂H₂₀N₄ONa [M+Na]⁺: 379.1545, found: 379.1532.

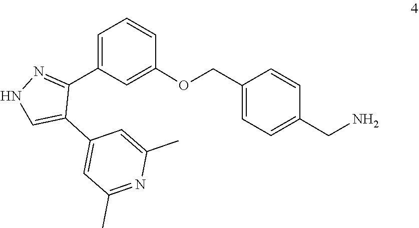 [SMILES: Cc1cc(-c2c[nH]nc2-c2cccc(OCc3ccc(CN)cc3)c2)cc(C)n1]

Compound 4, (4-((3-(4-(2,6-dimethylpyridin-4-yl)-1H-pyrazol-3-yl)phenoxy)methyl)phenyl)methanamine was synthesized according to general procedure VIII. This reaction was performed on a 0.26 mmol scale, providing the desired compound in 50% yield (50 mg). HRMS (ESI): m/z calc for C₂₄H₂₅N₄O [M+H]⁺: 385.2028, found: 385.2024.

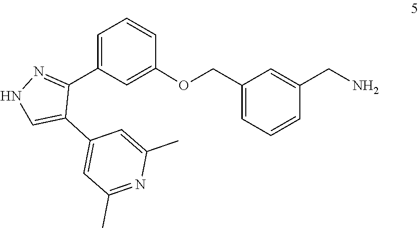 [SMILES: Cc1cc(-c2c[nH]nc2-c2cccc(OCc3cccc(CN)c3)c2)cc(C)n1]

Compound 5, (3-((3-(4-(2,6-dimethylpyridin-4-yl)-1H-pyrazol-3-yl)phenoxy)methyl)phenyl)methanamine was synthesized according to general procedure VIII. This reaction was performed on a 0.26 mmol scale, providing the desired compound in 48% yield (48 mg). HRMS (ESI): m/z calc for C₂₄H₂₅N₄O [M+H]⁺: 385.2028, found: 385.2014.

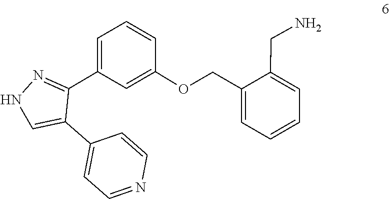 [SMILES: NCc1ccccc1COc1cccc(-c2n[nH]cc2-c2ccncc2)c1]

Compound 6, (2-((3-(4-(pyridin-4-yl)-1H-pyrazol-3-yl)phenoxy)methyl)phenyl)methanamine was synthesized according to general procedure VIII. This reaction was performed on a 0.93 mmol scale, providing the desired compound in 72% yield (238 mg). HRMS (ESI): m/z calc for C₂₂H₂₁N₄O [M+H]⁺: 357.1715, found: 357.1712.

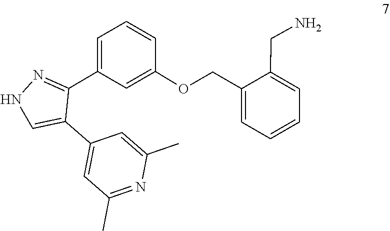 [SMILES: Cc1cc(-c2c[nH]nc2-c2cccc(OCc3ccccc3CN)c2)cc(C)n1]

Compound 7, (2-((3-(4-(2,6-dimethylpyridin-4-yl)-1H-pyrazol-3-yl)phenoxy)methyl)phenyl)methanamine was synthesized according to general procedure VIII. This reaction was performed on a 0.26 mmol scale, providing the desired compound in 51% yield (51 mg). HRMS (ESI): m/z calc for C₂₄H₂₅N₄O [M+H]⁺: 385.2028, found: 385.2031.

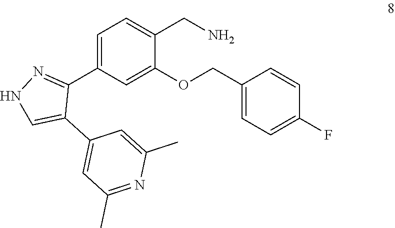 [SMILES: Cc1cc(-c2c[nH]nc2-c2ccc(CN)c(OCc3ccc(F)cc3)c2)cc(C)n1]

Compound 8, (4-(4-(2,6-dimethylpyridin-4-yl)-1H-pyrazol-3-yl)-2-((4-fluorobenzyl)oxy)phenyl)methanamine was synthesized according to general procedure VIII. This reaction was performed on a 0.36 mmol scale, providing the desired compound in 61% yield (89 mg). HRMS (ESI): m/z calc for C₂₄H₂₄FN₄O [M+H]⁺: 403.1934, found: 403.1923.

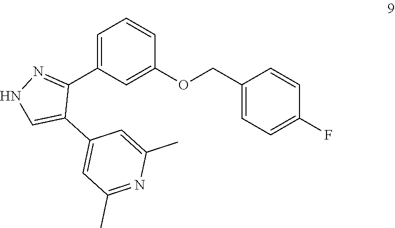 [SMILES: Cc1cc(-c2c[nH]nc2-c2cccc(OCc3ccc(F)cc3)c2)cc(C)n1]

Compound 9 (KAM-3-82), 4-(3-(3-((4-fluorobenzyl)oxy)phenyl)-1H-pyrazol-4-yl)-2,6-dimethylpyridine was synthesized according to general procedure VII. This reaction was performed on a 1.77 mmol scale, providing the desired compound in 74% yield (488 mg). HRMS (ESI): m/z calc for C₂₃H₂₁FN₃O [M+H]⁺: 374.1669, found: 374.1679.

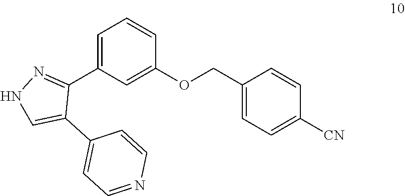 [SMILES: N#Cc1ccc(COc2cccc(-c3n[nH]cc3-c3ccncc3)c2)cc1]

Compound 10, 4-((3-(4-(pyridin-4-yl)-1H-pyrazol-3-yl)phenoxy)methyl)benzonitrile was synthesized according to general procedure VI. This reaction was performed on a 0.344 mmol scale, providing the desired compound in 56% yield (67.3 mg). HRMS (ESI): m/z calc for C₂₂H₁₇N₄O [M+H]⁺: 353.1402, found: 353.1395.

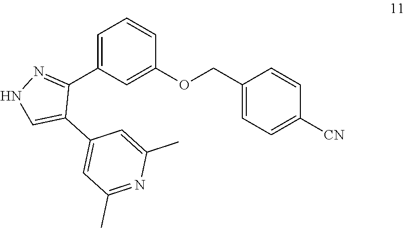 [SMILES: Cc1cc(-c2c[nH]nc2-c2cccc(OCc3ccc(C#N)cc3)c2)cc(C)n1]

Compound 11 (KAM-3-100), 4-((3-(4-(2,6-dimethylpyridin-4-yl)-1H-pyrazol-3-yl)phenoxy)methyl)benzonitrile was synthesized according to general procedure VII. This reaction was performed on a 0.81 mmol scale, providing the desired compound in 60% yield (185 mg). HRMS (ESI): m/z calc for C₂₄H₂₁N₄O [M+H]⁺: 381.1715, found: 381.1707.

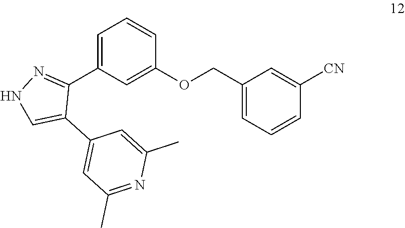 [SMILES: Cc1cc(-c2c[nH]nc2-c2cccc(OCc3cccc(C#N)c3)c2)cc(C)n1]

Compound 12 (Lolamycin), 3-((3-(4-(2,6-dimethylpyridin-4-yl)-1H-pyrazol-3-yl)phenoxy)methyl)benzonitrile was synthesized according to general procedure VII. This reaction was performed on a 2.0 mmol scale, providing the desired compound in 42% yield (324 mg). HRMS (ESI): m/z calc for C₂₄H₂₁N₄O [M+H]⁺: 381.1715, found: 381.1703.

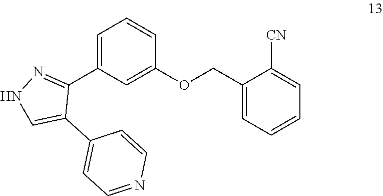 [SMILES: N#Cc1ccccc1COc1cccc(-c2n[nH]cc2-c2ccncc2)c1]

Compound 13, 2-((3-(4-(pyridin-4-yl)-1H-pyrazol-3-yl)phenoxy)methyl)benzonitrile was synthesized according to general procedure VI. The reaction was performed on a 1.2 mmol scale, providing the desired compound in 78% yield (326 mg). HRMS (ESI): m/z calc for C₂₂H₁₇N₄O [M+H]⁺: 353.1402, found: 353.1413.

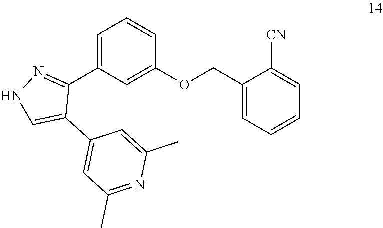 [SMILES: Cc1cc(-c2c[nH]nc2-c2cccc(OCc3ccccc3C#N)c2)cc(C)n1]

Compound 14, 2-((3-(4-(2,6-dimethylpyridin-4-yl)-1H-pyrazol-3-yl)phenoxy)methyl)benzonitrile was synthesized according to general procedure VII. This reaction was performed on a 2.0 mmol scale, providing the desired compound in 82% yield (624 mg). HRMS (ESI): m/z calc for C₂₄H₂₁N₄O [M+H]⁺: 381.1715, found: 381.1703.

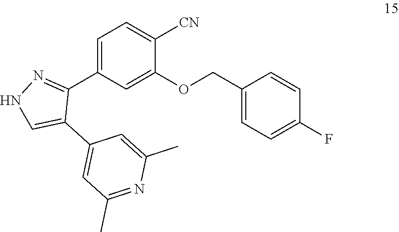 [SMILES: Cc1cc(-c2c[nH]nc2-c2ccc(C#N)c(OCc3ccc(F)cc3)c2)cc(C)n1]

Compound 15, 4-(4-(2,6-dimethylpyridin-4-yl)-1H-pyrazol-3-yl)-2-((4-fluorobenzyl)oxy)benzonitrile was synthesized according to general procedure VII. This reaction was performed on a 0.67 mmol scale, providing the desired compound in 55% yield (147 mg). HRMS (ESI): m/z calc for C₂₄H₂₀FN₄O [M+H]⁺: 399.1621, found: 399.1613.

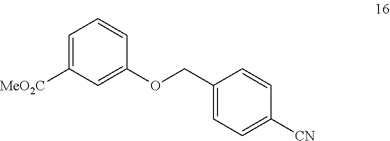 [SMILES: COC(=O)c1cccc(OCc2ccc(C#N)cc2)c1]

Compound 16, methyl 3-((4-cyanobenzyl)oxy)benzoate was synthesized according to general procedure I. This reaction was performed on a 6.9 mmol scale, providing the desired compound in 93% yield (1.42 g). ¹H NMR (500 MHz, Chloroform-d) δ 7.74-7.69 (m, 3H), 7.65 (dd, J=2.7, 1.5 Hz, 1H), 7.59 (d, J=8.1 Hz, 2H), 7.40 (t, J=8.0 Hz, 1H), 7.19 (ddd, J=8.2, 2.7, 1.0 Hz, 1H), 5.20 (s, 2H), 3.94 (s, 3H). ¹³C NMR (126 MHz, CDCl₃) δ 166.75, 158.15, 142.02, 132.47, 131.68, 129.67, 127.61, 122.79, 120.17, 118.65, 114.94, 111.91, 69.04, 52.30. HRMS (ESI): m/z calc for C₁₆H₁₄NO₃ [M+H]⁺: 268.0974, found: 268.0987.

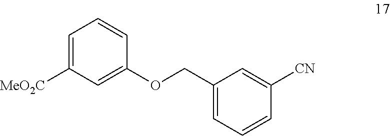 [SMILES: COC(=O)c1cccc(OCc2cccc(C#N)c2)c1]

Compound 17, methyl 3-((3-cyanobenzyl)oxy)benzoate was synthesized according to general procedure I. This reaction was performed on a 6.9 mmol scale, providing the desired compound in 98% yield (1.5 g). ¹H NMR (500 MHz, Chloroform-d) δ 7.76 (s, 1H), 7.70-7.66 (m, 2H), 7.63 (dd, J=4.7, 2.6 Hz, 2H), 7.51 (t, J=7.8 Hz, 1H), 7.38 (t, J=7.9 Hz, 1H), 7.17 (dd, J=8.3, 2.7 Hz, 1H), 5.14 (s, 2H), 3.92 (s, 3H). ¹³C NMR (126 MHz, CDCl₃) δ 166.89, 158.28, 138.37, 131.85, 131.80, 131.64, 130.87, 129.81, 129.59, 122.93, 120.34, 118.72, 115.02, 112.99, 68.94, 52.42. HRMS (ESI): m/z calc for C₁₆H₁₄NO₃ [M+H]⁺: 268.0974, found: 268.0973.

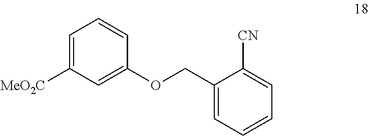 [SMILES: COC(=O)c1cccc(OCc2ccccc2C#N)c1]

Compound 18, methyl 3-((2-cyanobenzyl)oxy)benzoate was synthesized according to general procedure I. This reaction was performed on a 6.9 mmol scale, providing the desired compound in 98% yield (1.5 g). HRMS (ESI): m/z calc for C₁₆H₁₄NO₃ [M+H]⁺: 268.0974, found: 268.0973.

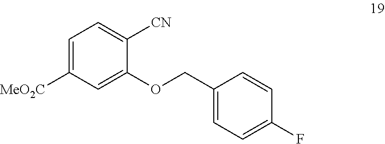 [SMILES: COC(=O)c1ccc(C#N)c(OCc2ccc(F)cc2)c1]

Compound 19, methyl 4-cyano-3-((4-fluorobenzyl)oxy)benzoate was synthesized according to general procedure I. This reaction was performed on a 6.8 mmol scale, providing the desired compound in 51% yield (793 mg). HRMS (ESI): m/z calc for C₁₆H₁₂NO₃NaF [M+Na]⁺: 308.0699, found: 308.0714.

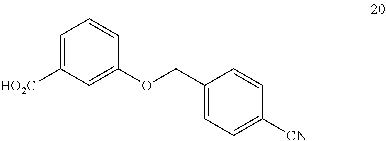 [SMILES: N#Cc1ccc(COc2cccc(C(=O)O)c2)cc1]

Compound 20, 3-((4-cyanobenzyl)oxy)benzoic acid was synthesized according to general procedure II. This reaction was performed on an 8.3 mmol scale, providing the desired compound in 75% yield (493 mg). ¹H NMR (500 MHz, DMSO-d₆) δ 7.89-7.86 (m, 2H), 7.66 (d, J=8.1 Hz, 2H), 7.56 (dt, J=7.6, 1.3 Hz, 1H), 7.53 (dd, J=2.9, 1.4 Hz, 1H), 7.43 (t, J=7.9 Hz, 1H), 7.28 (ddd, J=8.3, 2.8, 1.0 Hz, 1H), 5.29 (s, 2H). ¹³C NMR (126 MHz, DMSO) δ 167.49, 158.43, 143.15, 132.92, 130.31, 128.51, 127.62, 122.56, 120.20, 119.22, 115.34, 110.99, 68.85. HRMS (ESI): m/z calc for C₁₅H₁₂NO₃ [M+H]⁺: 254.0817, found: 254.0828.

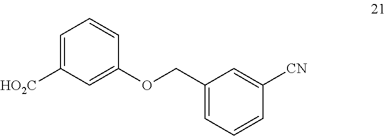 [SMILES: N#Cc1cccc(COc2cccc(C(=O)O)c2)c1]

Compound 21, 3-((3-cyanobenzyl)oxy)benzoic acid was synthesized according to general procedure II. This reaction was performed on a 5.6 mmol scale, providing the desired compound in 86% yield (1.22 g). ¹H NMR (499 MHz, Methanol-d₄) δ 7.86 (d, J=6.3 Hz, 1H), 7.83-7.78 (m, 1H), 7.73-7.68 (m, 1H), 7.68-7.63 (m, 2H), 7.60 (t, J=7.8 Hz, 1H), 7.45-7.39 (m, 1H), 7.29-7.24 (m, 1H), 5.22 (d, J=3.1 Hz, 2H). HRMS (ESI): m/z calc for C₁₅H₁₂NO₃ [M+H]⁺: 254.0817, found: 254.0807.

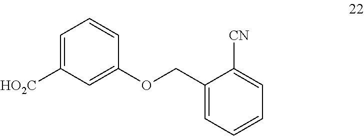 [SMILES: N#Cc1ccccc1COc1cccc(C(=O)O)c1]

Compound 22, 3-((2-cyanobenzyl)oxy)benzoic acid was synthesized according to general procedure II. This reaction was performed on a 5.6 mmol scale, providing the desired compound in 99% yield (1.41 g). HRMS (ESI): m/z calc for C₁₅H₁₂NO₃ [M+H]⁺: 254.0817.

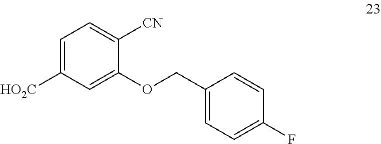 [SMILES: N#Cc1ccc(C(=O)O)cc1OCc1ccc(F)cc1]

Compound 23, 4-cyano-3-((4-fluorobenzyl)oxy)benzoic acid was synthesized according to general procedure II. This reaction was performed on a 2.8 mmol scale, providing the desired compound in 86% yield (650 mg).

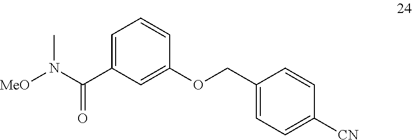 [SMILES: CON(C)C(=O)c1cccc(OCc2ccc(C#N)cc2)c1]

Compound 24, 3-((4-cyanobenzyl)oxy)-N-methoxy-N-methylbenzamide was synthesized according to general procedure III. This reaction was performed on a 2.0 mmol scale, providing the desired compound in 63% yield (378 mg). ¹H NMR (500 MHz, Chloroform-d) δ 7.66-7.63 (m, 2H), 7.52 (d, J=8.1 Hz, 2H), 7.33-7.27 (m, 2H), 7.24 (dd, J=2.7, 1.4 Hz, 1H), 7.03 (ddd, J=7.9, 2.7, 1.4 Hz, 1H), 5.13 (s, 2H), 3.52 (s, 3H), 3.32 (s, 3H). ¹³C NMR (126 MHz, CDCl₃) δ 169.33, 157.78, 142.21, 135.59, 132.41, 129.34, 127.61, 121.20, 118.67, 117.39, 114.31, 111.74, 68.97, 61.12, 33.82. HRMS (ESI): m/z calc for C₁₇H₁₇N₂O₃ [M+H]⁺: 297.1239, found: 297.1240.

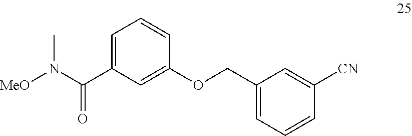 [SMILES: CON(C)C(=O)c1cccc(OCc2cccc(C#N)c2)c1]

Compound 25, 3-((3-cyanobenzyl)oxy)-N-methoxy-N-methylbenzamide was synthesized according to general procedure III. This reaction was performed on a 4.8 mmol scale, providing the desired compound in 60% yield (861 mg). ¹H NMR (500 MHz, Chloroform-d) δ 7.67 (d, J=1.7 Hz, 1H), 7.59 (dt, J=7.8, 1.5 Hz, 1H), 7.54 (dt, J=7.7, 1.4 Hz, 1H), 7.42 (t, J=7.8 Hz, 1H), 7.29-7.21 (m, 2H), 7.20-7.18 (m, 1H), 6.98 (ddd, J=8.0, 2.7, 1.3 Hz, 1H), 5.04 (s, 2H), 3.47 (s, 3H), 3.27 (s, 3H). ¹³C NMR (126 MHz, CDCl₃) δ 169.37, 157.78, 138.39, 135.53, 131.64, 131.54, 130.71, 129.46, 129.38, 121.25, 118.61, 117.48, 114.26, 112.76, 68.75, 61.15, 33.87. HRMS (ESI): m/z calc for C₁₇H₁₇N₂O₃ [M+H]⁺: 297.1239, found: 297.1229.

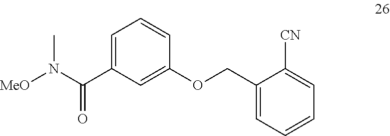 [SMILES: CON(C)C(=O)c1cccc(OCc2ccccc2C#N)c1]

Compound 26, 3-((2-cyanobenzyl)oxy)-N-methoxy-N-methylbenzamide was synthesized according to general procedure III. This reaction was performed on a 5.9 mmol scale, providing the desired compound in 90% yield (1.28 g). HRMS (ESI): m/z calc for C₁₇H₁₇N₂O₃ [M+H]⁺: 297.1239, found: 297.1230.

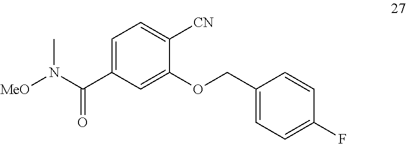 [SMILES: CON(C)C(=O)c1ccc(C#N)c(OCc2ccc(F)cc2)c1]

Compound 27, 4-cyano-3-((4-fluorobenzyl)oxy)-N-methoxy-N-methylbenzamide was synthesized according to general procedure III. This reaction was performed on a 2.4 mmol scale, providing the desired compound in 97% yield (733 mg).

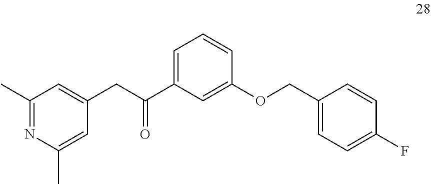 [SMILES: Cc1cc(CC(=O)c2cccc(OCc3ccc(F)cc3)c2)cc(C)n1]

Compound 28, 2-(2,6-dimethylpyridin-4-yl)-1-(3-((4-fluorobenzyl)oxy)phenyl)ethan-1-one was synthesized according to general procedure V. This reaction was performed on a 5.3 mmol scale, providing the desired compound in 38% yield (703 mg). HRMS (ESI): m/z calc for C₂₂H₂₁FNO₂ [M+H]⁺: 350.1556, found: 350.1552.

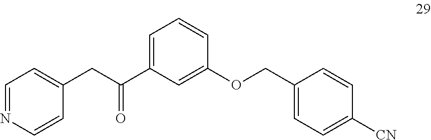 [SMILES: N#Cc1ccc(COc2cccc(C(=O)Cc3ccncc3)c2)cc1]

Compound 29, 4-((3-(2-(pyridin-4-yl)acetyl)phenoxy)methyl)benzonitrile was synthesized according to general procedure IV. This reaction was performed on a 1.3 mmol scale, providing the desired compound in 60% yield (290 mg). HRMS (ESI): m/z calc for C₂₁H₁₇N₂O₂ [M+H]⁺: 329.1290.

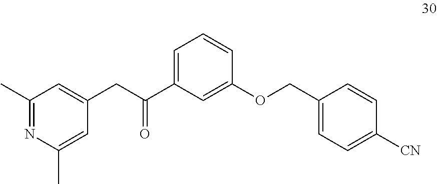 [SMILES: Cc1cc(CC(=O)c2cccc(OCc3ccc(C#N)cc3)c2)cc(C)n1]

Compound 30, 4-((3-(2-(2,6-dimethylpyridin-4-yl)acetyl)phenoxy)methyl)benzonitrile was synthesized according to general procedure V. This reaction was performed on a 1.2 mmol scale, providing the desired compound in 69% yield (290 mg). HRMS (ESI): m/z calc for C₂₃H₂₁N₂O₂ [M+H]⁺: 357.1603.

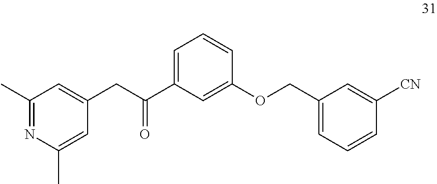 [SMILES: Cc1cc(CC(=O)c2cccc(OCc3cccc(C#N)c3)c2)cc(C)n1]

Compound 31 (KAM-3-170), 3-((3-(2-(2,6-dimethylpyridin-4-yl)acetyl)phenoxy)methyl)benzonitrile was synthesized according to general procedure V. This reaction was performed on a 2.9 mmol scale, providing the desired compound in 71% yield (733 mg). HRMS (ESI): m/z calc for C₂₃H₂₁N₂O₂ [M+H]⁺: 357.1603, found: 357.1596.

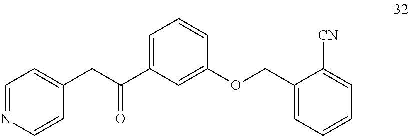 [SMILES: N#Cc1ccccc1COc1cccc(C(=O)Cc2ccncc2)c1]

Compound 32, 2-((3-(2-(pyridin-4-yl)acetyl)phenoxy)methyl)benzonitrile was synthesized according to general procedure IV. This reaction was performed on a 1.9 mmol scale, providing the desired product in 81% yield (502 mg). HRMS (ESI): m/z calc for C₂₁H₁₇N₂O₂ [M+H]⁺: 329.1290.

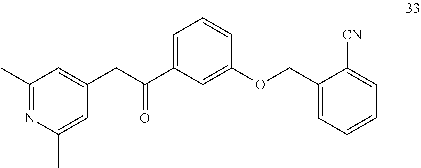 [SMILES: Cc1cc(CC(=O)c2cccc(OCc3ccccc3C#N)c2)cc(C)n1]

Compound 33, 2-((3-(2-(2,6-dimethylpyridin-4-yl)acetyl)phenoxy)methyl)benzonitrile was synthesized according to general procedure V. This reaction was performed on a 4.3 mmol scale, providing the desired compound in 46% yield (710 mg). HRMS (ESI): m/z calc for C₂₃H₂₁N₂O₂ [M+H]⁺: 357.1603.

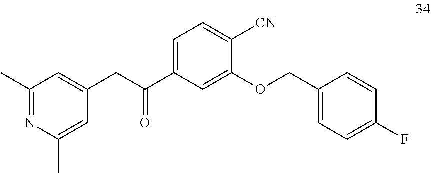 [SMILES: Cc1cc(CC(=O)c2ccc(C#N)c(OCc3ccc(F)cc3)c2)cc(C)n1]

Compound 34, 4-(2-(2,6-dimethylpyridin-4-yl)acetyl)-2-((4-fluorobenzyl)oxy)benzonitrile was synthesized according to general procedure V. This reaction was performed on a 2.3 mmol scale, providing the desired compound in 67% yield (585 mg).

### Example 4. Supplementary Data

TABLE 6
Antimicrobial assessment of amoxicillin, clindamycin, and lolamycin in a panel of human gut
commensal bacterial strains. Lolamycin was tested out to its solubility limit, which was 128 μg/mL. MICs
were performed in brain-heart infusion broth (Fisher Scientific; catalogue number: DF0418-17-7)
supplemented with 0.5% yeast extract, 0.1% L-cysteine hydrochloride, and 15 μg/mL hemin (BHI-ych).
MIC values were determined using the micro-dilution broth method as outlined by the Clinical and
Laboratory Standards Institute (CLSI. Methods for Antimicrobial Susceptibility Testing of Anaerobic
Bacteria. 9th ed. CLSI standard M11. Wayne, PA: Clinical and Laboratory Standards Institute; 2018) and
are listed in μg/mL. All experiments were performed in biological triplicate.

| Bacterial Strain | Amoxicillin | Clindamycin | Lolamycin |
|---|---|---|---|
| Gram-positive | | | |
| Streptococcus salivarius KLE 2370 | 0.125 | 2 | >128 |
| Streptococcus parasanguinis KLE 2375 | 64 | 0.06 | >128 |
| Enterococcus faecalis KLE 2341 | 0.25 | 1 | >128 |
| Bifidobacterium longum ATCC BAA-999 | 1 | 0.5 | >128 |
| Lactobacillus reuteri ATCC 23272 | 1 | 0.25 | >128 |
| Blautia producta ATCC 27340 | 1 | 1 | >128 |
| Gram-negative | | | |
| Bacteroides cellulosilyticus KLE 2342 | >128 | >128 | >128 |
| Bacteroides fragilis ATCC 25285 | 128 | 4 | >128 |
| Bacteroides vulgatus KLE 2303 | 2 | 16 | >128 |
| Faecalibacterium prausnitzii ATCC 27768 | 64 | 0.25 | >128 |
| Stenotrophomonas maltophilia ATCC 13637 | >128 | 0.5 | >128 |
| Morganella morganii ATCC 25839 | 32 | >128 | >128 |
| Proteus mirabilis ATCC 35659 | 0.008 | 0.125 | >128 |

TABLE 7
Sequence homology of E. coli (strain K12) LolCDE complex with other Gram-negatives.
Alignment: NCBI Blast-Blosum62; (GAVLI, FYW, CM, ST, KRH, DENQ, P).

| | LolC | LolD | LolE |
|---|---|---|---|
| Strain | Identity | Identity | Identity |
| Gram-negative pathogens | | | |
| Escherichia coli K12 | 100% | 100% | 100% |
| Shigella sonnei sp. | 99% | 99% | 99% |
| Salmonella typhimurium ATCC 14028 | — | 95% | 92% |
| Enterobacter cloacae sp. | 95% | 92% | 89% |
| Klebsiella pneumoniae ATCC BAA 2146 | 90% | 95% | 87% |
| Yersinia pestis sp. | 77% | 78% | 78% |
| Proteus mirabilis sp. | 61% | 68% | 63% |
| Vibrio cholerae sp. | 53% | 65% | 57% |
| Haemophilus influenzae sp. | 46% | 66% | 52% |
| Acinetobacter baumannii ATCC 19606 | 38% | 53% | 38% |
| Pseudomonas aeruginosa PAO1 | 37% | 57% | 37% |
| Moraxella catarrhalis sp. | 35% | 58% | 35% |
| Fusobacterium nucleatum ATCC 25586 | 25% | 42% | 20% |
| Human gut commensals | | | |
| Citrobacter sp. JGM124 | 82% | 92% | 80% |
| Hafnia alvei sp. | 74% | 74% | — |
| Morganella morganii sp. | 64% | 70% | 62% |
| Plesiomonas shigelloides sp. | 64% | 70% | 68% |
| Haemophilus parainfluenzae ATCC 33392 | 45% | 65% | 54% |
| Stenotrophomonas maltophilia sp. | 37% | 58% | — |
| Sutterella sp. | 37% | 56% | 37% |
| Odoribacter sp. | 36% | 46% | 28% |
| Desulfovibrio psychrotolerans sp. | 33% | 46% | 29% |
| Bilophila sp. | 31% | 49% | 28% |
| Alistipes indistinctus sp. | 27% | 50% | 27% |
| Prevotella sp. | 25% | 49% | 25% |
| Bacteroides vulgatus sp. | 25% | 44% | 25% |
| Parabacteroides merdae sp. | 25% | 44% | 25% |
| Bacteroides cellulosilyticus sp. | 24% | 44% | 24% |
| Bacteroides nordii sp. | 24% | 45% | 24% |
| Bacteroides fragilis ATCC 25285 | 23% | 44% | 23% |

TABLE 8
List of antibiotics and associated reference numbers
with clinically relevant levels of resistance in
E. coli, K. pneumoniae, and E. cloacae
clinical isolates assessed in Table 9-11.

| Antibiotic | |
|---|---|
| Reference | |
| Number | Antibiotic |
| 1 | Amikacin |
| 2 | Ampicillin |
| 3 | Ampicillin/sulbactam |
| 4 | Aztreonam |
| 5 | Cefazolin |
| 6 | Cefepime |
| 7 | Cefotaxime |
| 8 | Cefotaxime/clavulanic acid |
| 9 | Cefoxitin |
| 10 | Ceftazidime |
| 11 | Ceftazidime/avibactam |
| 12 | Ceftazidime/clavulanic acid |
| 13 | Ceftriaxone |
| 14 | Ciprofloxacin or |
| | Fluoroquinolone |
| 15 | Colistin |
| 16 | Doripenem |
| 17 | Ertapenem |
| 18 | Gentamicin |
| 19 | Imipenem |
| 20 | Imipenem + chelators |
| 21 | Levofloxacin |
| 22 | Meropenem |
| 23 | Piperacillin/tazobactam |
| 24 | Tetracycline |
| 25 | Tigecycline |
| 26 | Tobramycin |
| 27 | Trimethoprim/sulfamethoxazole |
| 28 | Minocycline |
| 29 | Streptomycin |

TABLE 9
Antimicrobial assessment of compound 2 and lolamycin in large panel of MDR clinical isolates
of E. coli. This data is depicted in FIG. 3. A full list of antibiotic resistance is provided. All compounds
were tested out to their solubility limits, which was 32 μg/mL for compound 2 or 128 μg/mL for
lolamycin. MICs were performed in cation-adjusted Mueller Hinton broth. MIC values were determined
using the micro-dilution broth method as outlined by the Clinical and Laboratory Standards Institute
(CLSI. Methods for Dilution Antimicrobial Susceptibility Tests for Bacteria that Grow Aerobically. 11^{th}
ed. CLSI standard M07. Wayne, PA: Clinical and Laboratory Standards Institute; 2018) and are listed in
μg/mL. All experiments were performed in biological triplicate.
MIC against Clinical Isolates of E. coli

| Strain | Drug Resistance | Colistin | 2 | Lolamycin |
|---|---|---|---|---|
| E. coli AR0048 | 1-7, 9-11, 13-15, 17-20, | 0.5 | >32 | 4 |
| | 22-23, 25-26 | | | |
| E. coli AR0055 | 1, 3-7, 9-11, 13-14, 16- | 1 | >32 | 2 |
| | 19, 21-24, 26-27 | | | |
| E. coli AR0058 | 2-7, 9-10, 13-14, 18, 21, | 0.5 | >32 | 2 |
| | 23-24, 27 | | | |
| E. coli AR0061 | 2-7, 10, 13, 16-19, 22- | 0.5 | >32 | 8 |
| | 24, 26-27 | | | |
| E. coli AR0067 | 2-3, 5, 9, 14, 21 | 1 | >32 | 2 |
| E. coli AR0069 | 2-3, 5-7, 9-11, 13, 16- | 0.5 | >32 | 2 |
| | 17, 19, 22-24, 26-27 | | | |
| E. coli AR0077 | Susceptible to all | 0.5 | 8 | 1 |
| E. coli AR0081 | 2-5, 7, 9-10, 13-14, 21 | 0.5 | 16 | 2 |
| E. coli AR0084 | 2, 10, 14, 21, 27 | 0.5 | >32 | 2 |
| E. coli AR0085 | 2-5, 7, 9-10, 13-14, 17, | 0.5 | >32 | 1 |
| | 21, 24, 27 | | | |
| E. coli AR0086 | 2-7, 9, 13-14, 18, 21, 24, | 0.5 | 8 | 0.5 |
| | 26-27 | | | |
| E. coli AR0089 | 2-5, 7, 9-10, 13, 24 | 0.5 | >32 | 2 |
| E. coli AR0104 | 2-7, 10, 13-14, 17, 21, | 0.5 | 16 | 1 |
| | 23-24, 27 | | | |
| E. coli AR0114 | 2-7, 9-10, 13-14, 16-17, | 0.25 | >32 | 2 |
| | 19, 21-23, 27 | | | |
| E. coli AR0118 | 1-7, 9-11, 13-14, 16-19, | 0.5 | 16 | 1 |
| | 21-23, 26-27 | | | |
| E. coli AR0119 | 1-7, 9-11, 13-14, 16-19, | 0.5 | >32 | 4 |
| | 20-23, 26-27 | | | |
| E. coli AR0128 | 1-7, 9-10, 12-14, 17-19, | 0.5 | 16 | 1 |
| | 21-22, 24, 26-27 | | | |
| E. coli AR0137 | 1-7, 9-11, 13-14, 16-19, | 1 | 16 | 2 |
| | 21-24, 26-27 | | | |
| E. coli AR0149 | 2-3, 5-7, 9-11, 13-14, | 0.5 | >32 | 2 |
| | 16-17, 19, 21-23 | | | |
| E. coli AR0150 | 2-7, 9-10, 13-14, 16-17, | 0.5 | 16 | 1 |
| | 19, 21-24, 27 | | | |
| E. coli AR0151 | 2-7, 9-10, 13-14, 16-17, | 0.5 | >32 | 2 |
| | 19, 21-24, 26-27 | | | |
| E. coli AR0162 | 2-7, 9-10, 13-14, 16-17, | 0.5 | >32 | 4 |
| | 19, 21-24 | | | |
| E. coli AR0346 | 1-7, 9-10, 13-14, 18, 21, | 4 | >32 | 2 |
| | 24, 26-27, 28 | | | |
| E. coli AR0348 | 2-7, 9-11, 13-14, 16-17, | 0.25 | >32 | 2 |
| | 19, 21-24, 27 | | | |
| E. coli AR0349 | 2-7, 10, 13-14, 21, 24, | 8 | >32 | 1 |
| | 26-27 | | | |
| E. coli AR0350 | 2-3, 5, 7, 9-10, 13-14, | 4 | >32 | 1 |
| | 21 | | | |
| E. coli AR0493 | 2-7, 9-10, 13-14, 21, 23- | 8 | >32 | 4 |
| | 24, 27 | | | |
| E. coli AR0494 | 2-3, 5, 14, 21, 24, 27-28 | 8 | >32 | 4 |
| E. coli AR0495 | 2-7, 10, 13-14, 21, 24, | 4 | >32 | 2 |
| | 27 | | | |
| E. coli AR0541 | 1-7, 10, 13-14, 21, 23, | 0.5 | >32 | 1 |
| | 24, 26-27 | | | |
| E. coli AR0543 | 2-7, 9-10, 13-14, 17-18, | 0.5 | >32 | 4 |
| | 21, 23-24, 26-27 | | | |
| E. coli AR0546 | 2-7, 9-10, 13-14, 16-19, | 0.5 | >32 | 0.5 |
| | 21-24, 26-27 | | | |
| E. coli AR0549 | 2-7, 9-10, 13-14, 16-17, | 0.25 | >32 | 2 |
| | 21-24, 26-27 | | | |
| E. coli AR0551 | 2-7, 9-10, 13-14, 18, 21, | 0.5 | >32 | 2 |
| | 24, 26-27 | | | |
| E. coli AR0559 | 1-7, 9-11, 13-14, 16-19, | 0.5 | >32 | 2 |
| | 21-24, 26-27 | | | |
| E. coli ATCC | 2-4, 6-7, 9-10, 13-14, | 0.25 | >32 | 2 |
| BAA-2340 | 16-18, 21-22, 24, 26-27, | | | |
| | see below | | | |
| E. coli ATCC | 1-4, 6-7, 9-10, 13-14, | 0.25 | >32 | 1 |
| BAA-2469 | 16-18, 21-24, 26-27, see | | | |
| | below | | | |
| E. coli ATCC | 2-4, 6-7, 9-10, 13-14, | 0.25 | >32 | 4 |
| BAA-2471 | 16-18, 21-24, 26-27, see | | | |
| | below | | | |
| E. coli | 14, ↑ AcrA | 0.25 | >32 | 2 |
| ELZ 4004 | | | | |
| E. coli | ↑ AcrA | 0.25 | 8 | 2 |
| ELZ 4017 | | | | |
| E. coli | None reported | 0.25 | >32 | 4 |
| ELZ 4042 | | | | |
| E. coli | None reported | 0.25 | >32 | 2 |
| ELZ 4346 | | | | |
| E. coli | 14, ↑ AcrA | 0.25 | 8 | 2 |
| ELZ 4072 | | | | |
| E. coli | 14, ↑ AcrA | 0.25 | 4 | 2 |
| ELZ 4081 | | | | |
| E. coli | 14, ↑ AcrA | 0.25 | >32 | 8 |
| ELZ 4139 | | | | |
| E. coli | 14, ↑ AcrA | 0.25 | >32 | 4 |
| ELZ 4239 | | | | |
| E. coli | 14, ↑ AcrA | 0.25 | >32 | 2 |
| ELZ 4277 | | | | |

For strain BAA-2340, resistance extends to cefalotin, cefuroxime, cefotetan, nalidixic acid, and cefpodoxime. Strains BAA-2469 and BAA-2471 are resistant to the aforementioned antibiotics in addition to ticarcillin. For ELZ strains, fluoroquinolone resistance has been observed in these clinical isolates, as well as increases in expression of acrA, a protein part of the AcrAB-TolC efflux system.

TABLE 10
Antimicrobial assessment of compound 2 and lolamycin in large panel of MDR clinical isolates
of K. pneumoniae. This data is depicted in FIG. 3. A full list of antibiotic resistance is provided. All
compounds were tested out to their solubility limits, which was 32 μg/mL for compound 2 or 128 μg/mL
for lolamycin. MICs were performed in cation-adjusted Mueller Hinton broth. MIC values were
determined using the micro-dilution broth method as outlined by the Clinical and Laboratory Standards
Institute (CLSI. Methods for Dilution Antimicrobial Susceptibility Tests for Bacteria that Grow
Aerobically. 11^{th} ed. CLSI standard M07. Wayne, PA: Clinical and Laboratory Standards Institute; 2018)
and are listed in μg/mL. All experiments were performed in biological triplicate.
MIC against Clinical Isolates of K. pneumoniae

| Strain | Drug Resistance | Colistin | 2 | Lolamycin |
|---|---|---|---|---|
| K. pneumoniae | 2-3, 5, 7, 9-11, 13, 17-19 | 0.5 | >32 | 0.5 |
| AR0034 | | | | |
| K. pneumoniae | 2-7, 9-10, 13-14, 16-17, | 0.5 | >32 | 0.5 |
| AR0039 | 19, 21, 23-24, 26-27 | | | |
| K. pneumoniae | 2-7, 9-11, 13-14, 16-19, | 8 | >32 | 1 |
| AR0040 | 21-24, 26-27 | | | |
| K. pneumoniae | 1-7, 9-10, 13-14, 16-19, | 0.5 | >32 | 0.5 |
| AR0041 | 21-24, 26-27 | | | |
| K. pneumoniae | 2-7, 9-10, 13-14, 16-19, | 0.5 | >32 | 4 |
| AR0042 | 21-23, 26-27 | | | |
| K. pneumoniae | 2-7, 10, 13-14, 17, 23 | 0.5 | >32 | 0.5 |
| AR0043 | | | | |
| K. pneumoniae | 2-7, 9-10, 13-14, 17-19, | 0.5 | >32 | 0.5 |
| AR0044 | 21-23, 26-27 | | | |
| K. pneumoniae | 2-7, 9-11, 13-14, 16-19, | 4 | >32 | 2 |
| AR0046 | 21-24, 26-27 | | | |
| K. pneumoniae | 2-7, 9-10, 13-14, 16-17, | 4 | >32 | 4 |
| AR0047 | 21-24, 26-27 | | | |
| K. pneumoniae | 1-7, 9-11, 13-15, 17-20, | 0.5 | >32 | 8 |
| AR0049 | 21-27 | | | |
| K. pneumoniae | 2-7, 9-10, 13-14, 16-19, | 1 | >32 | 4 |
| AR0066 | 21-24, 26-27 | | | |
| K. pneumoniae | 1-7, 9-11, 13-14, 16-19, | 0.5 | >32 | 4 |
| AR0068 | 21-23, 26-27 | | | |
| K. pneumoniae | 1-7, 9-10, 13-14, 16-19, | 0.5 | >32 | 1 |
| AR0075 | 21-24, 26-27 | | | |
| K. pneumoniae | 2-3, 5-7, 9-11, 13, 16, 18- | 0.5 | >32 | 0.5 |
| AR0076 | 19, 22-23, 26-27 | | | |
| K. pneumoniae | 1-7, 9-10, 13-14, 16-19, | 0.5 | >32 | 2 |
| AR0079 | 21-24, 26-27 | | | |
| K. pneumoniae | 2-7, 9-11, 13, 16-19, 22- | 0.5 | >32 | 0.5 |
| AR0080 | 23, 26 | | | |
| K. pneumoniae | 2-7, 9-10, 13-14, 21, 23- | 4 | >32 | 2 |
| AR0087 | 24 | | | |
| K. pneumoniae | 2-7, 9-10, 13-14, 16-19, | <0.25 | >32 | 4 |
| AR0098 | 21-23, 26-27 | | | |
| K. pneumoniae | 1-7, 9-11, 13-14, 16-19, | 4 | >32 | 2 |
| AR0106 | 21-24, 26-27 | | | |
| K. pneumoniae | 1-7, 9-10, 13-14, 16-19, | <0.25 | >32 | 0.25 |
| AR0107 | 21-23, 26-27 | | | |
| K. pneumoniae | 2-7, 9-10, 13-14, 16-18, | >8 | >32 | 8 |
| AR0109 | 21-24, 26-27 | | | |
| K. pneumoniae | 2-7, 9-10, 13-14, 16-19, | 0.5 | >32 | 4 |
| AR0112 | 21-23, 26-27 | | | |
| K. pneumoniae | 2-7, 9-10, 13-14, 16-19, | 0.5 | >32 | 2 |
| AR0113 | 21-23, 26-27 | | | |
| K. pneumoniae | 2-7, 9-10, 12-14, 16-17, | 0.5 | >32 | 8 |
| AR0115 | 19, 21-24, 26-27 | | | |
| K. pneumoniae | 2-7, 9-14, 16, 18-19, 21, | 1 | >32 | 4 |
| AR0117 | 23-24,26-27 | | | |
| K. pneumoniae | 2-7, 9-10, 13-15, 17-20, | 0.5 | >32 | 2 |
| AR0120 | 22-24 | | | |
| K. pneumoniae | 2-7, 9-10, 13-14, 17-19, | >4 | >32 | 4 |
| AR0125 | 21-22, 26-27 | | | |
| K. pneumoniae | 2-7, 9, 14, 17-18, 20, 23- | 0.5 | >32 | 0.5 |
| AR0126 | 24, 27 | | | |
| K. pneumoniae | 2-7, 9-10, 14-15, 17, 19, | 0.5 | >32 | 4 |
| AR0129 | 21-22, 24, 27 | | | |
| K. pneumoniae | 2-7, 9-11, 13-14, 16-19, | 0.5 | >32 | 2 |
| AR0135 | 21-24, 26-27 | | | |
| K. pneumoniae | 1-7, 9-10, 13-14, 16-19, | 2 | >32 | 1 |
| AR0138 | 21-24, 26-27 | | | |
| K. pneumoniae | 1-7, 9-11, 13-14, 16-19, | 2 | >32 | 2 |
| AR0139 | 21-24, 26-27 | | | |
| K. pneumoniae | 1-7, 9-10,13-14, 16-18, | 0.5 | >32 | 2 |
| AR0140 | 21-24, 26-27 | | | |
| K. pneumoniae | 1-7, 10, 13-14, 16-18, 21- | 1 | >32 | 0.5 |
| AR0141 | 24, 26-27 | | | |
| K. pneumoniae | 1-7, 9-10, 13-14, 17-19, | 0.5 | >32 | 0.5 |
| AR0142 | 23-24, 26-27 | | | |
| K. pneumoniae | 1-7, 9-11, 13-14, 16-19, | 1 | >32 | 4 |
| AR0143 | 21-24, 26-27 | | | |
| K. pneumoniae | 2-7, 9-10, 13-14, 16-17, | 0.5 | >32 | 2 |
| AR0145 | 19, 21-23, 26 | | | |
| K. pneumoniae | 2-7, 9-10, 13-14, 16-17, | 0.5 | >32 | 2 |
| AR0146 | 19, 21-24, 26-27 | | | |
| K. pneumoniae | 1-7, 9-10, 13-14, 16-19, | 1 | >32 | 4 |
| AR0148 | 21-24, 26-27 | | | |
| K. pneumoniae | 2-7, 9-10, 13-14, 16-17, | 0.5 | >32 | 0.5 |
| AR0152 | 19, 21-24, 26-27 | | | |
| K. pneumoniae | 1-7, 9-11, 13-14, 16-19, | 0.5 | >32 | 4 |
| AR0153 | 21-23, 26-27 | | | |
| K. pneumoniae | 2-7, 9-10, 13-14, 16-19, | 0.5 | >32 | 8 |
| AR0158 | 21-24, 27 | | | |
| K. pneumoniae | 2-3, 5, 7, 9, 16-17, 19, 22- | 1 | >32 | 0.5 |
| AR0160 | 23 | | | |
| K. pneumoniae | 2-7, 9-11, 13-14, 16-17, | <0.25 | >32 | 8 |
| AR0347 | 19, 21-24, 26-28 | | | |
| K. pneumoniae | 2-7, 9-10, 13-14, 17-18, | >8 | >32 | 2 |
| AR0542 | 21, 23, 24, 26-27 | | | |
| K. pneumoniae | 2-7, 9-10, 13-14, 16-17, | 0.5 | >32 | 2 |
| AR0548 | 19, 21-23, 27 | | | |
| K. pneumoniae | 2-7, 9-10, 13-14, 16-19, | <0.25 | >32 | 2 |
| AR0550 | 21-23, 26-27 | | | |
| K. pneumoniae | 2-7, 9-10, 13-14, 16-17, | 0.5 | >32 | 4 |
| AR0552 | 19, 21-24, 26-27 | | | |
| K. pneumoniae | 1-7, 9-10, 13-14, 16-19, | 0.5 | >32 | 0.5 |
| AR0553 | 21, 23, 26-27 | | | |
| K. pneumoniae | 1-7, 9-10, 13-14, 16-19, | 0.5 | >32 | 4 |
| AR0554 | 21-24, 26-27 | | | |
| K. pneumoniae | 1-7, 9-11, 13-14, 16-19, | 0.5 | >32 | 2 |
| AR0555 | 21-23, 26-27 | | | |
| K. pneumoniae ATCC | 1-4, 6-7, 9-10, 13-14, 16- | — | >32 | 8 |
| BAA-2146 | 19, 21-27, see below | | | |
| K. pneumoniae ATCC | 1-7, 9-10, 13-14 16-19, | — | >32 | 1 |
| BAA-2470 | 21-24, 26-27, see below | | | |
| K. pneumoniae ATCC | 1-7, 9-10, 13-14, 16-18, | — | >32 | 4 |
| BAA-2472 | 21-27, see below | | | |
| K. pneumoniae ATCC | 1-7, 9-10, 13-14, 16-19, | — | >32 | 4 |
| BAA-2473 | 21-27, see below | | | |
| K. pneumoniae ATCC | 1-6, 9-10, 13-14, 17, 19, | — | >32 | 4 |
| BAA-1705 | 21-22, 27, see below | | | |
| K. pneumoniae ATCC | 2-7, 9-10, 13-14, 16-17, | — | >32 | 4 |
| BAA-2342 | 19, 21-22, 27, see below | | | |

For strain BAA-2146, resistance extends to cefalotin, cefuroxime, cefotetan, nalidixic acid, cefpodoxime, ticarcillin, and nitrofurantoin. For strains BAA-2470, BAA-2472, BAA-2473, and BAA-2342, resistance extends to amoxicillin/clavulanic acid, piperacillin, ticarcillin, ticarcillin/clavulanic acid, cefalotin, cefuroxime, cefotetan, cefpodoxime, ceftizoxime, nalidixic acid, and nitrofurantoin. For strain BAA-1705, resistance extends to amoxicillin/clavulanic acid, piperacillin, and nitrofurantoin.

TABLE 11
Antimicrobial assessment of compound 2 and lolamycin in large panel of MDR clinical isolates
of E. cloacae. This data is depicted in FIG. 3. A full list of antibiotic resistance is provided. All
compounds were tested out to their solubility limits, which was 32 μg/mL for compound 2 or 128 μg/mL
for lolamycin. MICs were performed in cation-adjusted Mueller Hinton broth. MIC values were
determined using the micro-dilution broth method as outlined by the Clinical and Laboratory Standards
Institute (CLSI. Methods for Dilution Antimicrobial Susceptibility Tests for Bacteria that Grow
Aerobically. 11^{th} ed. CLSI standard M07. Wayne, PA: Clinical and Laboratory Standards Institute; 2018)
and are listed in μg/mL. All experiments were performed in biological triplicate.
MIC against Clinical Isolates of E. cloacae

| Strain | Drug Resistance | Colistin | 2 | Lolamycin |
|---|---|---|---|---|
| E. cloacae AR0032 | 2-5, 7, 9-10, 13, 18, 23, 27 | 0.5 | >32 | 2 |
| E. cloacae AR0038 | 1-14, 16-19, 21-24, 26-27 | 0.5 | >32 | 8 |
| E. cloacae AR0050 | 2-7, 9-10,13-14, 21, 23, 27 | 0.25 | 8 | 0.5 |
| E. cloacae AR0053 | 2-7, 9-10, 13-14, 16-19, 21-23, 26-27 | 0.5 | >32 | 4 |
| E. cloacae AR0060 | 2-5, 7, 9-10, 13, 23 | 0.5 | 16 | 1 |
| E. cloacae AR0065 | 2-7, 9-10, 13, 23 | 0.5 | 16 | 1 |
| E. cloacae AR0072 | 2-5, 7, 9-10, 13, 23 | 1 | >32 | 4 |
| E. cloacae AR0073 | 2-3, 5, 7, 9-10, 13 | 8 | >32 | 16 |
| E. cloacae AR0093 | 2-7, 9-10, 13-14, 17, 19, 22-23 | 0.5 | >32 | 2 |
| E. cloacae AR0132 | 2-5, 9, 17, 19, 22 | 1 | >32 | 1 |
| E. cloacae AR0136 | 2-7, 9-10, 13-14, 16-17, 19, 21-23, 26-27 | 0.5 | >32 | 4 |
| E. cloacae AR0154 | 2-7, 9-11, 13, 16-19, 23-24, 26-27 | 0.5 | 8 | 0.25 |
| E. cloacae AR0163 | 2-7, 9-10, 13-14, 16-19, 21-27 | 16 | >32 | 8 |
| E. cloacae AR0164 | 2-5, 7, 9-10, 13, 16-17, 19, 23 | 0.5 | >32 | 2 |
| E. cloacae AR0544 | 2-7, 9-10, 13-14, 16-17, 19, 21-23, 24, 26-27 | 0.25 | >32 | 4 |
| E. cloacae AR0545 | 2-7, 9-10, 13, 17-18, 23, 24, 26-27 | 0.5 | >32 | 1 |
| E. cloacae | 2-7, 9-10, 13-14, 16-19, 21-22, 24-27, see below | 0.5 | >32 | 16 |
| ATCC BAA-2341 | | | | |
| E. cloacae | 2-7, 9-10, 13-14, 16-19, 21-22, 24-27, see below | 0.25 | >32 | 4 |
| ATCC BAA-2468 | | | | |

For strains BAA-2341 and BAA-2468, resistance extends to amoxicillin/clavulanic acid, piperacillin, ticarcillin, ticarcillin/clavulanic acid, cefalotin, cefuroxime, cefotetan, cefpodoxime, ceftizoxime, nalidixic acid, and nitrofurantoin.

### Example 5. Pharmaceutical Dosage Forms

The following formulations illustrate representative pharmaceutical dosage forms that may be used for the therapeutic or prophylactic administration of a compound of a formula described herein, a compound specifically disclosed herein, or a pharmaceutically acceptable salt or solvate thereof (hereinafter referred to as ‘Compound X’):

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |
| (iii) Capsule | mg/capsule |
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |
| (iv) Injection 1 (1 mg/mL) | mg/mL |
| ‘Compound X’ (free acid form) | 1.0 |
| Dibasic sodium phosphate | 12.0 |
| Monobasic sodium phosphate | 0.7 |
| Sodium chloride | 4.5 |
| 1.0N Sodium hydroxide solution | q.s. |
| (pH adjustment to 7.0-7.5) | |
| Water for injection | q.s. ad 1 mL |
| (v) Injection 2 (10 mg/mL) | mg/mL |
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 0.1N Sodium hydroxide solution | q.s. |
| (pH adjustment to 7.0-7.5) | |
| Water for injection | q.s. ad 1 mL |
| (vi) Aerosol | mg/can |
| ‘Compound X’ | 20 |
| Oleic acid | 10 |
| Trichloromonofluoromethane | 5,000 |
| Dichlorodifluoromethane | 10,000 |
| Dichlorotetrafluoroethane | 5,000 |
| (vii) Topical Gel 1 | wt.% |
| ‘Compound X’ | 5% |
| Carbomer 934 | 1.25% |
| Triethanolamine | q.s. |
| (pH adjustment to 5-7) | 0.2% |
| Methyl paraben | |
| Purified water | q.s. to 100 g |
| (viii) Topical Gel 2 | wt.% |
| ‘Compound X’ | 5% |
| Methylcellulose | 2% |
| Methyl paraben | 0.2% |
| Propyl paraben | 0.02% |
| Purified water | q.s. to 100 g |
| (ix) Topical Ointment | wt.% |
| ‘Compound X’ | 5% |
| Propylene glycol | 1% |
| Anhydrous ointment base | 40% |
| Polysorbate 80 | 2% |
| Methyl paraben | 0.2% |
| Purified water | q.s. to 100 g |
| (x) Topical Cream 1 | wt.% |
| ‘Compound X’ | 5% |
| White bees wax | 10% |
| Liquid paraffin | 30% |
| Benzyl alcohol | 5% |
| Purified water | q.s. to 100 g |
| (xi) Topical Cream 2 | wt.% |
| ‘Compound X’ | 5% |
| Stearic acid | 10% |
| Glyceryl monostearate | 3% |
| Polyoxyethylene stearyl ether | 3% |
| Sorbitol | 5% |
| Isopropyl palmitate | 2% |
| Methyl Paraben | 0.2% |
| Purified water | q.s. to 100 g |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Compound X’. Aerosol formulation (vi) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A compound of Formula I: 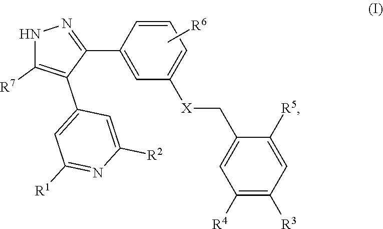
or a salt thereof;
wherein
R¹ and R² are each independently —CH₃, —(C₂-C₆)alkyl or —(C₃-C₆)cycloalkyl, or H;
R³ is H, halo, cyano, —(C₁-C₆)alkyl, or —(C₃-C₆)cycloalkyl;
R⁴ is cyano, H, halo, —(C₁-C₆)alkyl, or —(C₃-C₆)cycloalkyl;
R⁵ and R⁶ are each independently H, halo, cyano, —(C₁-C₆)alkyl, or —(C₃-C₆)cycloalkyl; and
R⁷ is H, —(C₁-C₆)alkyl, or —(C₃-C₆)cycloalkyl; and
X is O, S, NH, or NCH₃;
wherein any methyl, alkyl, or cycloalkyl moiety is optionally substituted; provided that when R¹ and R² are both H, R³ is not F and R³, R⁴, and R⁵ are not all H.

2. The compound of claim 1 wherein X is O.

3. The compound of claim 1 wherein R¹ and R² are CH₃.

4. The compound of claim 1 wherein R³ is H, F, CN, or CH₂NH₂.

5. The compound of claim 1 wherein R⁴ and R⁵ are each independently H, CN, or CH₂NH₂.

6. The compound of claim 1 wherein R⁶ is H, CN, or CH₂NH₂, wherein R⁶ is optionally at the position para to the pyrazole of Formula I.

7. The compound of claim 1 wherein one or two of R³, R⁴ and R⁵ are H; or R⁷ is H.

8. The compound of claim 1 represented by Formula II: 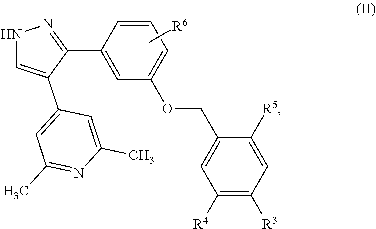
or a salt thereof.

9. The compound of claim 1 wherein the compound is lolamycin: 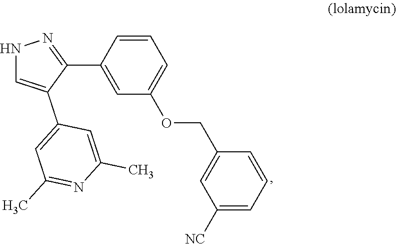 [SMILES: Cc1cc(-c2c[nH]nc2-c2cccc(OCc3cccc(C#N)c3)c2)cc(C)n1]
or a salt thereof.

10. The compound of claim 1 wherein the compound is:
(4-((3-(4-(pyridin-4-yl)-1H-pyrazol-3-yl)phenoxy)methyl)phenyl)methanamine (3);
(4-((3-(4-(2,6-dimethylpyridin-4-yl)-1H-pyrazol-3-yl)phenoxy)methyl)phenyl)methanamine (4);
(3-((3-(4-(2,6-dimethylpyridin-4-yl)-1H-pyrazol-3-yl)phenoxy)methyl)phenyl)methanamine (5);
(2-((3-(4-(pyridin-4-yl)-1H-pyrazol-3-yl)phenoxy)methyl)phenyl)methanamine (6);
(2-((3-(4-(2,6-dimethylpyridin-4-yl)-1H-pyrazol-3-yl)phenoxy)methyl)phenyl)methanamine (7);
(4-(4-(2,6-dimethylpyridin-4-yl)-1H-pyrazol-3-yl)-2-((4-fluorobenzyl)oxy)phenyl)methanamine (8);
4-(3-(3-((4-fluorobenzyl)oxy)phenyl)-1H-pyrazol-4-yl)-2,6-dimethylpyridine (9);
4-((3-(4-(pyridin-4-yl)-1H-pyrazol-3-yl)phenoxy)methyl)benzonitrile (10);
4-((3-(4-(2,6-dimethylpyridin-4-yl)-1H-pyrazol-3-yl)phenoxy)methyl)benzonitrile (11);
3-((3-(4-(2,6-dimethylpyridin-4-yl)-1H-pyrazol-3-yl)phenoxy)methyl)benzonitrile (12, lolamycin);
2-((3-(4-(pyridin-4-yl)-1H-pyrazol-3-yl)phenoxy)methyl)benzonitrile (13);
2-((3-(4-(2,6-dimethylpyridin-4-yl)-1H-pyrazol-3-yl)phenoxy)methyl)benzonitrile (14); or
4-(4-(2,6-dimethylpyridin-4-yl)-1H-pyrazol-3-yl)-2-((4-fluorobenzyl)oxy)benzonitrile (15).

11. A method for treating a patient infected with Gram-negative bacteria comprising administering to the patient a therapeutically effective amount of a compound of claim 1,
wherein the compound is an inhibitor of the localization of lipoprotein CDE (LolCDE) complex of the ATP Binding Cassette transporter, and the infected patient is thereby treated.

12. The method of claim 11 wherein the Gram-negative bacteria is Escherichia coli, Klebsiella pneumoniae, Enterobacter cloacae, or Salmonella typhimurium.

13. The method of claim 11 wherein the compound is not antimicrobial against intestinal bacteria.

14. The method of claim 11 wherein the compound kills 50% of the Gram-negative bacteria at about 1 mg/mL to about 3 mg/mL; or
the compound kills 90% of the Gram-negative bacteria at about 7 mg/mL to about 9 mg/mL.

15. The method of claim 11 wherein the compound is 3-((3-(4-(2,6-dimethylpyridin-4-yl)-1H-pyrazol-3-yl)phenoxy)methyl)benzonitrile (12, lolamycin).
